(19) United States
(12) Patent Application Publication
(10) Pub. No.: US 2010/0172994 A1
(43) Pub. Date: Jul. 8, 2010

(54) Nanoparticles for Protection of Cells from Oxidative Stress

(75) Inventors: Wolfgang M. Sigmund, Gainesville, FL (US); Yi-Yang Tsai, Brandon, FL (US); Ioannis Constantinidis, Gainesville, FL (US); Jenny Dorley, Gainesville, FL (US); Jose Antonio Oca-Cossio, Gainesville, FL (US); Carol Ann Sweeney, Micanopy, FL (US); Nicholas Edward Simpson, Alachua, FL (US); Mark A. Atkinson, Gainesville, FL (US)

Correspondence Address: SALIWANCHIK LLOYD & SALIWANCHIK;A PROFESSIONAL ASSOCIATION, PO Box 142950, GAINESVILLE, FL, 32614, US

(73) Assignee: UNIVERSITY OF FLORIDA RESEARCH FOUNDATION, INC., GAINESVILLE, FL (US)

(21) Appl. No.: 12/445,299

(22) PCT Filed: Nov. 23, 2007
(86) PCT No.: PCT/US2007/085470

§ 371 Date: Feb. 8, 2010

Related U.S. Application Data
(60) Provisional application No. 60/860,646, filed on Nov. 22, 2006.

(51) Int. Cl. A61K 9/14; A61K 35/12; A61K 33/24

(52) U.S. Cl. 424/489; 424/93.7; 424/617; 435/375; 977/773; 977/904

(57) ABSTRACT

The present invention concerns metal oxide semiconductor nanoparticles with free radical scavenging activity, compositions comprising such nanoparticles, methods for their use, and methods for their production. In one aspect, the invention concerns a method for enhancing the survival or viability of transplanted cells, comprising administering an effective amount of metal oxide semiconductor nanoparticles to a target anatomical site of a subject before, during, or after administration of transplant cells to the subject. Preferably, the metal oxide nanoparticle is a cerium oxide (ceria) nanoparticle.

### CROSS-REFERENCE TO RELATED APPLICATION

The present application claims the benefit of U.S. Provisional Application Ser. No. 60/860,646, filed Nov. 22, 2006, which is hereby incorporated by reference herein in its entirety, including any figures, tables, nucleic acid sequences, amino acid sequences, and drawings.

### BACKGROUND OF THE INVENTION

Cell transplantation therapy is a potentially powerful tool in the treatment of diseases for which there are currently no practical cures. Theoretically, the replacement of defective cells by healthy cells offers the possibility of alleviating the devastating symptoms for many such diseases including Parkinson's disease, stroke, Alzheimer's disease, spinal cord injury, type I diabetes, cirrhosis of the liver and factor 8 hemophilia. The success of the “Edmonton protocol”, which resulted in a 100% cure rate for human Type I diabetes following the transplantation of islet allografts (Shapiro, A. M. et al. N Engl J Med, 2000, 343:230-238), attests to this attractive potential. Clearly, allo- and xenografted cells can restore function to dysfunctional tissues in experimental animal models of disease (for review see Emerich, D. F. et al. Cell Transplant, 2003, 12:335-349)).

Despite improvements in the management of individuals with type 1 diabetes, the disorder remains a leading cause of blindness, kidney and heart disease, limb amputation, and other disease associated complications. For many years, pancreatic islet cell transplantation has been proposed as an ideal treatment to alleviate insulin dependence among those with type 1 diabetes. Despite recent improvements to the procedure, many obstacles remain including the necessity for patients to utilize potentially deleterious immunosuppressive drugs. One problem may be that the immunosuppressive drugs cannot undo the underlying autoimmune response that originally destroyed a patient's original islets, [4] while another critical issue involves the loss in islet viability and function post-transplantation.

Free radicals are known causes of various ailments and thus, the ability to reduce their intracellular concentrations can significant improve human health. Cells synthesize enzymes such as superoxide dismutase and catalase that can scavenge such free radicals. However, exogenous introduction of these enzymes is not beneficial because they cannot be readily taken up by the cells.

Of the challenges facing the field of cell transplantation, one of the principle concerns has been that of transplant cell viability in vivo. Transplantation protocols that increase the viability of the transplanted cells would facilitate cell therapy.

### BRIEF SUMMARY OF THE INVENTION

The present invention concerns metal oxide semiconductor nanoparticles with free radical scavenging activity, compositions comprising such nanoparticles, methods for their use, and methods for their production.

In one aspect, the invention provides citrate-coated metal oxide nanoparticles having free radical scavenging activity, which are useful for increasing the survival or viability of cells in vitro and in vivo. Preferably, the nanoparticle is a citrate-coated cerium oxide nanoparticle.

In another aspect, the invention concerns a method for enhancing the survival or viability of cells in vitro (e.g., ex vivo), comprising culturing, incubating, or otherwise contacting in vitro one or more target cell types with metal oxide nanoparticles having free radical scavenging activity, for a time sufficient to enhance or increase cell survival or viability in vitro.

In another aspect, the invention concerns a method for enhancing the survival or viability of endogenous cells at a target anatomical site of a subject (in vivo), comprising administering an effective amount of metal oxide semiconductor nanoparticles to the target anatomical site of the subject. Preferably, the metal oxide nanoparticles are citrate coated. In one embodiment, the target anatomical site is the pancreas.

In another aspect, the invention concerns a method for enhancing the survival or viability of transplanted cells, comprising administering an effective amount of metal oxide semiconductor nanoparticles to a target anatomical site of a subject before, during, or after administration of transplant cells to the subject. In one embodiment, the nanoparticles are administered simultaneously with the transplant cells and within the same composition.

The metal oxide nanoparticle can be any metal oxide nanoparticle that scavenges free radicals and is non-toxic in the amount administered. For example, the metal oxide can be zinc oxide yttrium oxide, zirconium oxide, bismuth oxide, or cadmium oxide. Preferably, the metal oxide nanoparticle is a cerium oxide (ceria) nanoparticle.

Preferably, the nanoparticles are citrate-coated.

In another embodiment, the nanoparticles are doped. Preferably, the nanoparticles are doped such that fluorescence is conferred to the nanoparticle. In one embodiment, the nanoparticles are Erbium-doped. Doped, fluorescent nanoparticles are useful in nanomedicine and bio-imaging. Since the conventional Q-Dots for bio-imaging are toxic due to their nature of free radical generation, tEr-doped CeO₂ nanoparticles, for example, provide advantages (they are also free radical scavengers) in the same types of applications. Er-doped CeO₂ nanoparticles have similar surface properties with CeO₂ nanoparticles, and can be used to monitor the allocations of CeO₂ nanoparticles in cells as well as organs. These Er—CeO₂ nanoparticles are crucial for the future developments in this project. Thus doped, fluorescent, metal oxide nanoparticles can be administered to cells in vitro or in vivo, and the fluorescent nanoparticles can be visualized (imaged), using any necessary equipment.

In one embodiment, the transplant cells are pancreatic cells, such as islet cells.

The nanoparticles are typically less than 20 nanometers, in diameter. In preferred embodiments, the nanoparticles are equal to or less than 10 nanometers in diameter. More preferably, the nanoparticles are within the range of 3 nanometers and 7 nanometers in diameter.

Another aspect of the invention concerns a composition comprising nanoparticles of the invention and cells of one or more types. Preferably, the nanoparticles are citrate-coated. More preferably, the nanoparticles are citrate-coated cerium oxide nanoparticles. The cells and/or composition can be therapeutic, intended for transplantation. Alternatively, the cells and/or composition can be intended for production of molecules that may be subsequently harvested from the cells.

Another aspect of the invention concerns a cell preservation fluid comprising metal oxide nanoparticles of the invention and one or more cell preservation agents. Preferably, the nanoparticles are citrate-coated. More preferably, the nanoparticles are citrate-coated cerium oxide nanoparticles.

In accordance with the invention, free radical scavenging particles can be used as agents to increase the numbers of preserved cells in transplantation of cells; to enhance the viability of transplanted organs both prior to transplantation, i.e., during transport and in vivo after transplantation; to enhance viability and/or function of cell based tissue substitutes; to improve cells' or organisms' lifespans; to improve animals' or humans' health; to improve animals' or humans' lifespan; for anti-aging; for anti-inflammation; and cosmetics; and, optionally, can be used in conjunction with other effective agents.

### BRIEF DESCRIPTION OF THE DRAWINGS

FIGS. 1A-1C show transmission electron micrographs (TEM) of cerium oxide nanoparticles. The nanoparticles are 3 nanometers to 5 nanometers in size (FIG. 1A). They are weakly flocculated after synthesis (FIG. 1B), and can be dispersed in solution (FIG. 1C).

FIG. 2 shows a bar graph indicating the ability of CeO₂ nanoparticles to scavenge reactive oxygen species in human islets. Notable is the increase in the DCF/PI ratio when islets were exposed to 100 μM H₂O₂ and the reduction in the ratio when 200 μM CeO₂ nanoparticles were provided to the islets.

FIG. 3 shows a TEM of ceria nanoparticles via reverse micelle synthesis.

FIGS. 4A and 4B show TEM of several βTC-tet cells, with FIG. 4A obtained at 2,000×, and FIG. 4B at 25,000×, showing one of these cells.

FIGS. 5A and 5B show the behavior of βTC-tet cells in the presence and absence of ceria nanoparticles. FIG. 5A shows glucose consumption rates (GSR) and FIG. 5B shows insulin secretion rates (ISR) at 0 mM and 20 mM glucose.

FIGS. 6A and 6B show temporal changes in the number of βTC-tet cells as a function of CeO₂ concentrations (FIG. 6A), and percent viable βTC-tet cells after 4 days of exposure to CeO₂ containing media (Day 5 in FIG. 6A).

FIGS. 7A and 7B show rates of oxygen consumption (OCR) (FIG. 7A), and insulin secretion (ISR) (FIG. 7B) by βTC-tet cells after 4 days of exposure to CeO₂ containing media.

FIG. 8 shows percent viable βTC-tet cells following 4 days of serum deprivation. The error bars represent the standard deviation of the mean based on measurements generated by three independent flasks. Cells were exposed to CeO₂ containing media for 4 days during the expansion of the culture but not during the 4 days of serum deprivation.

FIG. 9 shows percent viable βTC-tet cells following 1 day of H₂O₂ exposure. The error bars represent the standard deviation of the mean based on measurements generated by two independent flasks. Cells were exposed to CeO₂ containing media for 4 days during the expansion of the culture but not during the subsequent 24 hours of H₂O₂ exposure.

FIG. 10 shows a bar graph indicating the ability of CeO₂ nanoparticles to scavenge ROS in human islets. The y-axis represent a ratio of the fluorescent signal generated by DCF (an index of intracellular ROS) and popidium iodine (PI, an index of cell numbers). Human islets were incubated for 3 days in CMRL media containing 50, 100, or 200 μm CeO₂ nanoparticles, followed by a 2 hour exposure to 50 μM H₂O₂.

FIGS. 11A-11H show data from radical scavenging experiments. HEK; DCF 48-hour CeO₂ pretreatment (from 20 mM dispersion) at 0, 400, and 1500 uM; and 20-hour HQ exposure at 500 uM. FIG. 11A: No CeO₂; No HQ; 13%. FIG. 11B: 400 uM CeO₂; No HQ; 9%. FIG. 11C: 1500 uM CeO₂; No HQ; 5%. FIG. 11D: No CeO₂; 500 uM HQ; 55%. FIG. 11E: 400 uM CeO₂; 500 uM HQ; 40%. FIG. 11F: 1500 uM CeO₂; 500 uM HQ; 20%. FIG. 11G: 400 uM CeO₂; 500 uM HQ; 45%. FIG. 11H: 1500 uM CeO₂; 500 uM HQ; 19%.

FIGS. 12A-12H show data from radical scavenging experiments. HEK; Caspase 48-hour CeO₂ pretreatment (from 10 mM dispersion) at 0, 400, and 1500 uM; and 20-hour HQ exposure at 500 uM. FIG. 12A: No CeO₂; No HQ; 12%. FIG. 12B: 400 uM CeO₂; No HQ; 11%; FIG. 12C: 1500 uM CeO₂; No HQ; 7%. FIG. 12D: No CeO₂; 500 uM HQ; 52%. FIG. 12E: 400 uM CeO₂; 500 uM HQ; 56%. FIG. 12F: 1500 uM CeO₂; 500 uM HQ; 23%. FIG. 12G: 400 uM CeO₂; 500 uM HQ; 56%. FIG. 12H: 1500 uM CeO₂; 500 uM HQ; 21%.

FIGS. 13A-13H show data from radical scavenging experiments. HEK; PI 48-hour CeO₂ pretreatment (from 10 mM dispersion) at 0, 400, and 1500 uM; and 20-hour HQ exposure at 500 uM. FIG. 13A: No CeO₂; No HQ; 46%. FIG. 13B: 400 uM CeO₂; No HQ; 40%. FIG. 13C: 1500 uM CeO₂; No HQ; 20%. FIG. 13D: No CeO₂; 500 uM HQ; 83%. FIG. 13E: 400 uM CeO₂; 500 uM HQ; 77%. FIG. 13F: 1500 uM CeO₂; 500 uM HQ; 55%. FIG. 13G: 400 uM CeO₂; 500 uM HQ; 79%. FIG. 13H: 1500 uM CeO₂; 500 uM HQ; 63%.

FIGS. 14A-14L show data from radical scavenging experiments. 042007 INS-1, TC-TET, Fibroblast, HQ and CeO₂. INS-1, 500 uM HQ for 20 hours, 1500 uM CeO₂ for 48 hours. FIG. 14A: DCF; No CeO₂; No HQ; 15%. FIG. 14B: DCF; No CeO₂; 500 uM HQ; 56%. FIG. 14C: DCF; 1500 uM CeO₂; No HQ; 14%. FIG. 14D: DCF; 1500 uM CeO₂; 500 uM HQ; 16%. FIG. 14E: Caspase; No CeO₂; No HQ; 16%. FIG. 14F: Caspase; No CeO₂; 500 uM HQ; 38%. FIG. 14G: Caspase; 1500 uM CeO₂; No HQ; 8%. FIG. 14H: Caspase; 1500 uM CeO₂; 500 uM HQ; 28%. FIG. 14I: PI; No CeO₂; No HQ; 38%. FIG. 14J: PI; No CeO₂; 500 uM HQ; 77%. FIG. 14K: PI; 1500 uM CeO₂; No HQ; 14%. FIG. 14L: PI; 1500 uM CeO₂; 500 uM HQ; 30%.

FIGS. 15A-15L show data from radical scavenging experiments. 042007 INS-1, TC-TET, Fibroblast, HQ and CeO₂. TC-TET, 500 uM HQ for 20 hours, 1500 uM CeO₂ for 48 hours. FIG. 15A: DCF; No CeO₂; No HQ; 18%. FIG. 15B: DCF; No CeO₂; 500 uM HQ; 51%. FIG. 15C: DU; 1500 uM CeO₂; No HQ; 22%. FIG. 15D: DCF; 1500 uM CeO₂; 500 uM HQ; 23%, FIG. 15E: Caspase; No CeO₂; No HQ; 30%. FIG. 15F: Caspase; No CeO₂; 500 uM HQ; 49%. FIG. 15G: Caspase; 1500 uM CeO₂; No HQ; 9%. FIG. 15H: Caspase; 1500 uM CeO₂; 500 uM HQ; 19%. FIG. 15I: PI; No CeO₂; No HQ; 31%. FIG. 15J: PI; No CeO₂; 500 uM HQ; 38%. FIG. 15K: PI; 1500 uM CeO₂; No HQ; 6%. FIG. 15L: PI; 1500 uM CeO₂; 500 uM HQ; 16%.

FIGS. 16A-16L show data from radical scavenging experiments. 042007 INS-1, TC-TET, Fibroblast, HQ and CeO₂. Fibroblast, 500 uM HQ for 20 hours, 1500 uM CeO₂ for 48 hours. FIG. 16A: DCF; No CeO₂; No HQ; 6%. FIG. 16B: DCF; No CeO₂; 500 uM HQ; 3%. FIG. 16C: DCF; 1500 uM CeO₂; No HQ; 11%. FIG. 16D: DCF; 1500 uM CeO₂; 500 uM HQ; 4%. FIG. 16E: Caspase; No CeO₂; No HQ; 8%. FIG. 16F: Caspase; No CeO₂; 500 uM HQ; 8%. FIG. 16G: Caspase; 1500 uM CeO₂; No HQ; 3%. FIG. 16H: Caspase; 1500 uM CeO₂; 500 uM HQ; 8%. FIG. 16I: PI; No CeO₂; No HQ; 40%. FIG. 16J: PI; No CeO₂; 500 uM HQ; 6%. FIG. 16K: PI; 1500 uM CeO₂; No HQ; 16%. FIG. 16L: PI; 1500 uM CeO₂; 500 uM HQ; 7%.

FIGS. 17A and 17B show peroxide radical scavenging efficiency of 7 nm commercial CeO₂ and synthetic CexZr1-xO2 (x=0, 0.2, 0.4, 0.6, 0.7, 0.8, 1.0) nanoparticles. FIG. 17A shows peroxide concentration in the presence of nanoparticles in variant of time. FIG. 17B shows natural logarithm values of peroxide concentration by initial peroxide concentration based on the results from FIG. 17A. The slope of series curves in FIG. 17B indicates the free radical scavenging rate of these nanoparticles.

FIG. 18 shows comparison of peroxide radical scavenging efficiencies of commercial CeO₂ nanoparticles and synthesized CexZr1-xO2 (x=0, 0.2, 0.4, 0.6, 0.7, 0.8, 1.0) nanoparticles. The scavenging efficiency of synthesized CeO₂ nanoparticles is set to 1, while the scavenging efficiency of CexZr1-xO2 nanoparticles increases when zirconium was doped into lattice. The scavenging efficiency was improved up to 4× when 30% of zirconium ions substituted cerium ions. The efficiency was then reduced when more than 40% of cerium ions were substituted. The efficiency of 7 nm commercial CeO₂ nanoparticles was only one fourth of synthesized nanoparticles. The results showed that we are able to synthesized CexZr1-xO2 nanoparticles of 16× higher radical scavenging efficiency than commercial CeO₂ nanoparticles. In addition, the CexZr1-xO2 nanoparticles that we developed exhibited 4× higher free radical scavenging efficiency than those nanoparticles developed in UCF.

FIGS. 19A and 19B show fluorescent images of Erbium doped CeO₂ nanoparticles (FIG. 19A). The Er-doped CeO₂ nanoparticles are fluorescent in nature, and the fluorescence can be excited light of 488 nm wavelength and detected at 540-660 nm wavelength (FIG. 19B). These Er-doped CeO₂ nanoparticles are applicable in nanomedicine, Q-Dots, and bio-imaging. Since the conventional Q-Dots for bio-imaging are toxic due to their nature of free radical generation, these Er-doped CeO₂ nanoparticles provide advantages (they are also free radical scavengers) in the same types of applications. These Er-doped CeO₂ nanoparticles have similar surface properties with CeO₂ nanoparticles, and can be used to monitor the allocations of CeO₂ nanoparticles in cells as well as organs. These Er—CeO₂ nanoparticles are crucial for the future developments in this project.

### DETAILED DESCRIPTION OF THE INVENTION

Cell therapy is a potentially powerful tool in the treatment of many disorders including leukemia, immune deficiencies, autoimmune diseases and diabetes. In one aspect, the invention provides a citrate-coated metal oxide nanoparticle with free radical scavenging activity, which is useful for increasing the survival or viability, and potentially function, of cells in vitro and in vivo. Preferably, the nanoparticle is a citrate-coated cerium oxide nanoparticle.

In another aspect, the invention concerns a method for enhancing the survival or viability of endogenous cells at a target anatomical site of a subject, comprising administering an effective amount of metal oxide semiconductor nanoparticles to the target anatomical site of the subject. In one embodiment, the target anatomical site is the pancreas. In another embodiment, the target anatomical site is the skin, and free radical scavenging nanoparticles are administered to enhance survival or viability of resident skin cells. For example, the free radical scavenging nanoparticles can be administered to the skin as a cream, lotion, or gel formulation, as an anti-aging agent.

In another aspect, the invention concerns a method for enhancing the survival or viability of transplanted cells, comprising administering an effective amount of metal oxide semiconductor nanoparticles to a target anatomical site of a subject before, during, or after administration of transplant cells to the subject. In one embodiment, the nanoparticles are administered simultaneously with the transplant cells and within the same composition. In one embodiment, the target anatomical site is the pancreas and the transplant cells are pancreatic islet cells. In another embodiment, the target anatomical site is the skin and the transplant cells are skin cells (e.g., as a skin graft).

The metal oxide nanoparticle can be any metal oxide nanoparticle that scavenges free radicals and is non-toxic in the amount administered. For example, the metal oxide can be zinc oxide yttrium oxide, zirconium oxide, bismuth oxide, or cadmium oxide. Preferably, the metal oxide nanoparticle is a cerium oxide (ceria) nanoparticle.

Optionally, the free radical scavenging nanoparticles include a targeting agent useful for targeting the nanoparticles to specific cell types or tissues.

Optionally, the transplantation methods of the invention further comprises administering one or more immunosuppressive agents to the subject. Optionally, the nanoparticles and cells are administered concurrently with one or more second therapeutic modalities, e.g., symptomatic treatment, high dose immunosuppressive therapy. Such methods are known in the art and can include administration of agents useful for treating an autoimmune disorder, e.g., NSAIDs (including selective COX-2 inhibitors); other antibodies, e.g., anti-cytokine antibodies, e.g., antibodies to IFN-alpha, IFN-gamma, and/or TNF-alpha; heat shock proteins (e.g., as described in U.S. Pat. No. 6,007,821); immunosuppressive drugs (such as corticosteroids, e.g., prednisolone and methyl prednisolone; cyclophosphamide; azathioprine; mycophenolate mofetil (MMF); cyclosporin and tacrolimus; methotrexate; or cotrimoxazole) and therapeutic cell preparations, e.g., subject-specific cell therapy.

The transplantation methods described herein can also be used to enhance transplant cell survival or viability in a transplant recipient. For example, the methods can be used in a wide variety of tissue and organ transplant procedures, e.g., the methods can be used to enhance transplant cell viability in a recipient of a graft of cells, e.g., stem cells such as bone marrow and/or of a tissue or organ such as pancreatic islets, liver, kidney, heart, lung, skin, muscle, neuronal tissue, stomach, and intestines. Thus, the transplantation methods of the invention can be applied in treatments of diseases or conditions that entail cell, tissue or organ transplantation (e.g., liver transplantation to treat hypercholesterolemia, transplantation of muscle cells to treat muscular dystrophy, or transplantation of neuronal tissue to treat Huntington's disease or Parkinson's disease). The transplantation methods of the invention involve administering to a subject in need of transplantation: a) an effective amount of free radical scavenging nanoparticles (systemically administered or locally administered); and b) donor cells. The donor cells can be isolated cells or comprise tissue or an organ, e.g., liver, kidney, heart, lung, skin, muscle, neuronal tissue, stomach and intestines.

In some embodiments, the transplanted cells comprise pancreatic islets. Accordingly, the invention encompasses a method for treating diabetes by pancreatic islet cell transplantation. The method comprises administering to a subject in need of treatment: a) an effective amount of free radical scavenging nanoparticles; and b) donor pancreatic islet cells. The nanoparticles can be administered to the recipient prior to, simultaneously with, or after administration of the pancreatic islets.

In some embodiments, the recipient is then treated with a regimen of immune-suppressing drugs to suppress rejection of the donor cells (e.g., isolated cells, tissue, or organ). Standard regimens of immunosuppressive treatment are known. Tolerance to donor antigen can be evaluated by standard methods, e.g., by MLR assays.

In some embodiments, the donor is a living, viable human being, e.g., a volunteer donor, e.g., a relative of the recipient. In some embodiments, the donor is no longer living, or is brain dead, e.g., has no brain stem activity. In some embodiments, the donor tissue or organ is cryopreserved. In some embodiments, the donor is one or more non-human mammals, e.g., an inbred or transgenic pig, or a non-human primate.

Mammalian species which benefit from the methods of the invention include, but are not limited to, primates, such as apes, chimpanzees, orangutans, humans, monkeys; domesticated animals (e.g., pets) such as dogs, cats, guinea pigs, hamsters, Vietnamese pot-bellied pigs, rabbits, and ferrets; domesticated farm animals such as cows, buffalo, bison, horses, donkey, swine, sheep, and goats; exotic animals typically found in zoos, such as bear, lions, tigers, panthers, elephants, hippopotamus, rhinoceros, giraffes, antelopes, sloth, gazelles, zebras, wildebeests, prairie dogs, koala bears, kangaroo, opossums, raccoons, pandas, hyena, seals, sea lions, elephant seals, otters, porpoises, dolphins, and whales. As used herein, the terms “patient”, “individual”, “subject”, “host”, and “recipient” are interchangeable and intended to include such human and non-human mammalian species.

Typically, the dose of donor cells (transplant cells) administered to a subject, particularly a human, in the context of the present invention should be sufficient to achieve a therapeutic response in the patient over a reasonable time frame. One skilled in the art will recognize that dosage will depend upon a variety of factors including the condition of the animal, the body weight of the animal, as well as the severity and stage of disease, if present.

The cells and/or nanoparticles are preferably administered to a patient in an amount effective to provide a therapeutic benefit. A “therapeutically effective amount” can be that amount effective to treat a pathological condition. For purposes of the subject invention, the terms “treat” or “treatment” include preventing, inhibiting, reducing the occurrence of and/or ameliorating the physiological effects of the pathological condition to be treated. Preferably, the cells are administered to the subject in an amount within the range of about 10⁴ to about 10¹⁰ cells. More preferably, the cells are administered to the subject in an amount within the range of about 10⁷ to about 10¹⁰ cells. Doses of cells can be determined by one of ordinary skill in the art, with consideration given to such factors as cell survival rate following administration, the number of cells necessary to induce a physiologic response in the normal state, and the species of the subject.

Patients in need of treatment using the methods of the present invention can be identified using standard techniques known to those in the medical profession.

The donor cells (transplant cells) can be administered as cell therapy to alleviate the symptoms of a wide variety of disease states and pathological conditions, in various stages of pathological development. For example, donor cells can be used to treat acute disorders (e.g., stroke or myocardial infarction), and administered acutely, subacutely, or in the chronic state. Similarly, the donor cells can be used to treat chronic disorders (e.g., Parkinson's disease, diabetes, or muscular dystrophy), and administered preventatively and/or prophylactically, early in the disease state, in moderate disease states, or in severe disease states. For example, the donor cells can be administered to a target site or sites on or within a patient in order to replace or compensate for the patient's own damaged, lost, or otherwise dysfunctional cells. This includes infusion of the cells into the patient's bloodstream. The cells to be administered can be cells of the same cell type as those damaged, lost, or otherwise dysfunctional, or a different cell type or types. For example, insulin-producing pancreatic islet beta cells supplemented with other types of cells of the subject invention can be administered to the liver (Goss, J. A., et al., Transplantation, Dec. 27, 2002, 74(12):1761-1766). As used herein, patients “in need” of the donor cells (transplant cells) include those desiring elective surgery, such as elective cosmetic surgery.

The donor cells (transplant cells) can be administered as autografts, syngeneic grafts; allografts, and xenografts, for example. The donor cells administered to the subject may be obtained from any of the aforementioned species in which the cells are found. As used herein, the term “graft” refers to one or more cells intended for implantation within a human or non-human subject. Hence, the graft can be a cellular or tissue graft, for example.

The nanoparticles and donor cells can be administered to a subject by any route, such as intravascularly, intracranially, intracerebrally, intramuscularly, intradermally, intravenously, intraocularly, orally, nasally, topically, or by open surgical procedure, depending upon the anatomical site or sites to which the cells and nanoparticles are to be delivered. Preferably, the nanoparticles and donor cells are administered to the subject by the same route. Preferably, the nanoparticles are administered at the same anatomic site as the donor cells, or immediately adjacent thereto. Donor cells can be administered in an open manner, as in the heart during open heart surgery, or in the brain during stereotactic surgery, or by intravascular interventional methods using catheters going to the blood supply of the specific organs, or by interventional methods such as intrahepatic artery injection of pancreatic cells for diabetics.

Pharmaceutical compositions used in the methods of the invention can be formulated according to known methods for preparing pharmaceutically useful compositions. Formulations are described in a number of sources which are well known and readily available to those skilled in the art. For example, Remington's Pharmaceutical Sciences (Martin E W, 1995, Easton Pa., Mack Publishing Company, 19^{th} ed.) describes formulations which can be used in connection with the subject invention. Formulations suitable for parenteral administration include, for example, aqueous sterile injection solutions, which may contain antioxidants, buffers, bacteriostats, and solutes which render the formulation isotonic with the blood of the intended recipient; and aqueous and nonaqueous sterile suspensions which may include suspending agents and thickening agents. The formulations may be presented in unit-dose or multi-dose containers, for example sealed ampoules and vials, and may be stored in a freeze dried (lyophilized) condition requiring only the condition of the sterile liquid carrier, for example, water for injections, prior to use. It should be understood that in addition to the ingredients particularly mentioned above, the formulations of the subject invention can include other agents conventional in the art having regard to the type of formulation in question.

The nanoparticles and/or donor cells may also be administered intravenously or intraperitoneally by infusion or injection. Solutions of the nanoparticles and/or cells can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the nanoparticles or donor cells in the desired amount in the appropriate solvent with any of the other various ingredients enumerated above, as required, followed by filter sterilization.

As used herein, the terms “administering,” “introducing,” “delivering,” “placing”, “applying”, “implanting”, and “transplanting” and grammatical variations thereof are used interchangeably herein and refer to the placement of nanoparticles and/or cells into a subject in vivo by any method or route that results in at least partial localization of the cells at a desired site, or to the placement of nanoparticles and/or cells to a target location in vitro. Thus, “transplant” or “transplanted” cells include those that have been grown in vitro, and may have been genetically modified, as well as the transplantation of material extracted from another organism. The source of the transplant cells can be the intended recipient, or a donor of the same species or different species as that of the intended recipient. The cells can be administered by any appropriate route which results in delivery to a desired location in the subject where at least a portion of the cells or components of the cells remain viable. The period of viability of the transplant cells after co-administration of the transplant cells and nanoparticles to a subject can be as short as a few hours, e.g., twenty-four hours, to a few days, to as long as several years.

As used herein, the terms “treating” and “treatment” include reducing or alleviating at least one adverse effect or symptom of a disease or disorder.

As used herein, “therapeutically effective dose of cells” refers to an amount of cells that are sufficient to bring about a beneficial or desired clinical effect. The dose could be administered in one or more administrations. However, the precise determination of what would be considered an effective dose may be based on factors individual to each patient, including, but not limited to, the patient's age, size, type or extent of disease, stage of the disease, route of administration of the cells, the type or extent of supplemental therapy used, ongoing disease process and type of treatment desired (e.g., aggressive vs. conventional treatment). An “effective amount” or “effective dose” of nanoparticles is that amount that increases the survival or viability of co-administered cells relative to their survival or viability in the absence of the nanoparticles.

The terms “recombinant host cells”, “host cells”, “cells”, “cell lines”, and other such terms denoting microorganisms or higher eukaryotic cell lines refer to cells which can be, or have been, used as recipients for recombinant vectors or other transfer DNA, immaterial of the method by which the DNA is introduced into the cell or the subsequent disposition of the cell. The terms include the progeny of the original cell that has been transfected. The donor cells (transplant cells) can be those of primary cultures, or cells which have been passaged one or more times, for example. In a preferred embodiment, the donor cells (transplant cells) are cells of cell lines.

The donor cells (transplant cells) may be genetically modified or non-genetically modified cells. The term “genetic modification” as used herein refers to the stable or transient alteration of the genotype of a cell by intentional introduction of exogenous nucleic acids by any means known in the art (including for example, direct transmission of a polynucleotide sequence from a cell or virus particle, transmission of infective virus particles, and transmission by any known polynucleotide-bearing substance) resulting in a permanent or temporary alteration of genotype. The nucleic acids may be synthetic, or naturally derived, and may contain genes, portions of genes, or other useful polynucleotides. A translation initiation codon can be inserted as necessary, making methionine the first amino acid in the sequence.

The donor cells (transplant cells) may be transformed or non-transformed cells. The terms “transfection” and “transformation” are used interchangeably herein to refer to the insertion of an exogenous polynucleotide into a host cell, irrespective of the method used for the insertion, the molecular form of the polynucleotide that is inserted, or the nature of the cell (e.g., prokaryotic or eukaryotic). The insertion of a polynucleotide per se and the insertion of a plasmid or vector comprised of the exogenous polynucleotide are included. The exogenous polynucleotide may be directly transcribed and translated by the cell, maintained as a nonintegrated vector, for example, a plasmid, or alternatively, may be stably integrated into the host genome.

The cells used in the methods and compositions of the invention can range in plasticity from totipotent or pluripotent stem cells (e.g., adult or embryonic), precursor or progenitor cells, to highly specialized or mature cells, such as those of the central nervous system (e.g., neurons and glia) or islets of Langerhans. Stem cells can be obtained from a variety of sources, including fetal tissue, adult tissue, umbilical cord blood, peripheral blood, bone marrow, and brain, for example. Methods and markers commonly used to identify stem cells and to characterize differentiated cell types are described in the scientific literature (e.g., Stern Cells Scientific Progress and Future Research Directions, Appendix E1-E5, report prepared by the National Institutes of Health, June, 2001). The list of adult tissues reported to contain stem cells is growing and includes bone marrow, peripheral blood, umbilical cord blood, brain, spinal cord, dental pulp, blood vessels, skeletal muscle, epithelia of the skin and digestive system, cornea, retina, liver, and pancreas.

As will be understood by one of skill in the art, there are over 200 cell types in the human body. It is believed that the methods and compositions of the invention can be used to increase the survival or viability of any of these cells types when they are administered for therapeutic or other purposes. For example, any cell arising from the ectoderm, mesoderm, or endoderm germ cell layers can be administered and their survival and viability enhanced by the free radical scavenging nanoparticles of the invention. Such cells include, but are not limited to, neurons, glial cells (astrocytes and oligodendrocytes), muscle cells (e.g., cardiac, skeletal), chondrocytes, fibroblasts, melanocytes, Langerhans cells, keratinocytes, endothelial cells, epithelial cells, pigment cells (e.g., melanocytes, retinal pigment epithelial (RPE) cells, iris pigment epithelial (IPE) cells), hepatocytes, microvascular cells, pericytes (Rouget cells), blood cells (e.g., erythrocytes), cells of the immune system (e.g., B and T lymphocytes, plasma cells, macrophages/monocytes, dendritic cells, neutrophils, eosinophils, mast cells), thyroid cells, parathyroid cells, pituitary cells, pancreatic cells (e.g., insulin-producing β cells, glucagon-producing α cells, somatostatin-producing δ cells, pancreatic polypeptide-producing cells, pancreatic ductal cells), stromal cells, Sertoli cells, adipocytes, reticular cells, rod cells, and hair cells. Likewise, the compositions of the invention can include any combination of cells and free radical scavenging nanoparticles. Other examples of cell types for which the survival or viability can be increased with free radical scavenging nanoparticles include those disclosed by Spier R. E. et al., eds., (2000) The Encyclopedia of Cell Technology, John Wiley & Sons, Inc., and Alberts B. et al., eds., (1994) Molecular Biology of the Cell, 3^{rd} ed., Garland Publishing, Inc., e.g., pages 1188-1189, each of which are incorporated herein by reference in their entirety. Table 1 contains a non-exhaustive list of cells that may be de-differentiated, re-programmed, and/or stabilized in accordance with the present invention. In one embodiment, the cells are non-neural cells. In another embodiment, the cells are non-neuronal cells. In another embodiment, the cells are non-retinal cells. In one embodiment, the cells are insulin producing cells. The cells can be “normal” cells, lacking any biochemical and/or genetic abnormalities associated with that disease. Alternatively, there may be circumstances in which it is desired to enhance the survival or viability non-normal cells (e.g., cancer cells) in vitro or in vivo (e.g., in cell culture or in an animal model for research purposes).

In another aspect, the invention concerns a method for enhancing the survival or viability of cells in vitro (e.g., ex vivo), comprising culturing, incubating, or otherwise contacting in vitro one or more target cell types with nanoparticles having free radical scavenging activity, for a time sufficient to enhance or increase cell survival or viability in vitro. In one embodiment, the cells are non-neural cells. In another embodiment, the cells are non-neuronal cells. Various culturing methods known in the art can be used to contact the target cells with the free radical scavenging nanoparticles (or composition containing the nanoparticles) for a period of time, and in such a way that the survival and viability of the target cells is enhanced in vitro and/or in vivo. Contacting can be carried out under in vitro conditions, such as in suspension cultures or by allowing cells to adhere to a fixed substrate, or under in vivo conditions. For example, using a container with large growth surfaces, such as round bottles, cells can be grown in a confluent monolayer. The bottles can be rotated or agitated in motorized devices to keep the cells in suspension (e.g., the “roller flask” technique). Roller culture apparatus and similar devices are commercially available (WHEATON SCIENCE PRODUCTS).

The cells can be cultured in the presence of free radical scavenging nanoparticles as heterogeneous mixtures of cells or cell types, or clonally, for example. A cell is said to be clonally derived or to exhibit clonality if it was generated by the division of a single cell and is genetically identical to that cell. Purified populations (clonal lines) are particularly useful for in vitro cell response studies, efficient production of specific biomolecules, and cell transplant therapy, because the exact identity of the cells' genetic capabilities and functional qualities are readily identified. In order to increase the survival or viability of target cells in vitro or in vivo, the target cells can be exposed to the free radical scavenging nanoparticles disclosed herein in vitro and/or in vivo by various methods known in the art for cell treatment. Furthermore, various techniques of isolating, culturing, and characterizing cells can be utilized to carryout the method of the subject invention, including those techniques described in Freshney R. I., ed., (2000), Culture of Animal Cells: A Manual of Basic Technique, Fourth edition, Wiley-Liss, New York. For example, the target cells can be exposed to free radical scavenging nanoparticles in the presence, or absence, of various substances, such as serum or other trophic factors.

A wide variety of media, salts, media supplements, and products for media formulation can be utilized to produce the continuous cell lines of the subject invention, depending upon the particular type of target cell. Examples of these substances include, but are not limited to, carrier and transport proteins (e.g., albumin), biological detergents (e.g., to protect cells from shear forces and mechanical injury), biological buffers, growth factors, hormones, hydrosylates, lipids (e.g., cholesterol), lipid carriers, essential and non-essential amino acids, vitamins, sera (e.g., bovine, equine, human, chicken, goat, porcine, rabbit, sheep), serum replacements, antibiotics, antimycotics, and attachment factors. These substances can be present in various classic and/or commercially available media, which can also be utilized with the subject invention. Examples of such media include, but are not limited to, Ames' Medium, Basal Medium Eagle (BME), Click's Medium, Dulbecco's Modified Eagle's Medium (DMEM), DMEM/Nutrient Mixture F12 Ham, Fischer's Medium, Minimum Essential Medium Eagle (MEM), Nutrient Mixtures (Ham's). Waymouth Medium, and William's Medium E.

The effects of the free radical scavenging nanoparticles, or other synthetic or biological agents, on the cells can be identified on the basis of significant difference relative to control cultures with respect to criteria such as the ratios of expressed phenotypes, cell viability, function, and alterations in gene expression. Physical characteristics of the cells can be analyzed by observing cell morphology and growth with microscopy. Increased or decreased levels of proteins, such as enzymes, receptors and other cell surface molecules, amino acids, peptides, and biogenic amines can be analyzed with any technique known in the art which can identify the alteration of the level of such molecules. These techniques include immunohistochemistry, using antibodies against such molecules, or biochemical analysis. Such biochemical analysis includes protein assays, enzymatic assays, receptor binding assays, enzyme-linked immunosorbent assays (ELISA), electrophoretic analysis, analysis with high performance liquid chromatography (HPLC), Western blots, and radioimmune assays (RIA). Nucleic acid analysis, such as Northern blots and polymerase chain reaction (PCR) can be used to examine the levels of mRNA coding for these molecules, or for enzymes which synthesize these molecules. Alternatively, cells treated with free radical scavenging nanoparticles can be transplanted into an animal, and their survival and biochemical and immunological characteristics examined.

Cells treated with free radical scavenging nanoparticles can be used as a platform for growing virus particles for vaccine production or other purposes. For example, human cervical epithelium can be proliferated in culture and used to support human papilloma virus in the development of a vaccine. In addition, fetal kidney cells are commonly used for the production of several different vaccines.

Cells treated with free radical scavenging nanoparticles according to the methods of the subject invention can have a naturally occurring or induced defect, such that the cells provide an in vitro model of disease. As described above with respect to normal cells, these cells can be used to test effects of synthetic or biological agents in a disease model. For example, the establishment of stable, in vitro models of the nervous system will provide an important tool to rapidly and accurately address various neurological disorders. Therefore, a cell line treated according to the methods of the subject invention can be obtained having similar dysfunction mechanisms as the originating tissues, and which would serve as a model to study potential therapies and/or further alterations of the cell function.

Depending upon cell type, differentiation of the cells can be induced by any method known in the art that activates the cascade of biological events that lead to cell growth, before, during, or after exposure to the free radical scavenging nanoparticles. For example, cells can be induced to differentiate by plating the cells on a fixed substrate, such as a flask, plate, or coverslip, or a support of collagen, fibronectin, laminin, or extracellular matrix preparation such as MATRIGEL (Collaborative Research), or removal of conditioned medium. Cells can be incubated in dishes and on cover slips coated with MATRIGEL to allow gellification and subsequently seeded onto the treated surface (Cardenas, A. M. et al., Neuroreport., 1999, 10:363-369). Differentiation can be induced by transfer to GM with 1% bovine serum and 10 μg/ml of both insulin and transferrin, wherein differentiating media is F12/D supplemented with 1% bovine serum and 1% stock supplement (Liberona, J. L. et al., Muscle & Nerve, 1998, 21:902-909).

Cells can be stimulated to differentiate by contact with one or more differentiation agents (e.g., trophic factors, hormonal supplements), such as forskolin, retinoic acid, putrescin-transferrin, cholera toxin, insulin-like growth factor (IGF), transforming growth factor (e.g., TGF-α, TGF-β), tumor necrosis factor (TNF), fibroblast growth factor (FGF), epidermal growth factor (EGF), granulocyte macrophage-colony stimulating factor (GM-CSF), hepatocyte growth factor (HGF), hedgehog, vascular endothelial growth factor (VEGF), thyrotropin releasing hormone (TRH), platelet derived growth factor (PDGF), sodium butyrate, butyric acid, cyclic adenosine monophosphate (cAMP), cAMP derivatives (e.g., dibutyryl cAMP, 8-bromo-cAMP) phosphodiesterase inhibitors, adenylate cyclase activators, prostaglandins, ciliary neurotrophic factor (CNTF), brain-derived neurotrophic factor (BDNF), neurotrophin 3, neurotrophin 4, interleukins (e.g., IL-4), interferons (e.g., interferon-gamma), leukemia inhibitory factor (LIF), potassium, amphiregulin, dexamethasone (glucocorticoid hormone), isobutyl 3-methyulxanthine, somatostatin, lithium, and growth hormone.

The subject invention provides a ready source of cells for research, including pharmacological studies for the screening of various agents, and toxicologic studies for the cosmetic and pharmaceutical industries. The cells of the subject invention can be used in methods for determining the effect of a synthetic or biological agent on cells. The term “biological agent” refers to any agent of biological origin, such as a virus, protein, peptide, amino acid, lipid, carbohydrate, nucleic acid, nucleotide, drug, pro-drug, or other substance that may have an effect on cells, whether such effect is harmful, beneficial, or otherwise. Thus, the cells of the present invention can be used for screening agonists and antagonists of compounds and factors that affect the various metabolic pathways of a specific cell, for example. The choice of cell will depend upon the particular agent being tested and the effects one wishes to achieve.

It should be understood that the free radical scavenging nanoparticles can be administered to a subject to increase survival or viability of donor cells, regardless of the purposes the donor cells were administered. Thus, the method of the invention may also be utilized when it is desired to transplant cells for non-therapeutic purposes (i.e., for purposes other than cell therapy). For example, the method of the invention can be used to provide enhanced survival or viability to transplanted cells in a subject, wherein the transplanted cells were administered to the subject for research purposes. In this case, the transplanted donor cells need not provide any therapeutic effect. Such research purposes include, but are not limited to, the study of cell migration and differentiation, and the cellular decisions that occur during cell determination and differentiation. Furthermore, the ability of the transplanted cells to express endogenous or heterologous genes in a human or non-human subject in vivo can be studied.

The nanoparticles and/or donor cells can include one or more labels. For example, the donor cells can be labeled to track their migration and/or differentiation within the subject's tissue in vivo or ex vivo (see, for example, Cahill, K. et al. Transplantation, 2004, 78(11):1626-1633; and Lekic, P. C. et al. Anal Rec, 2001, 262(2):193-202; Bulte, J. W. et al. Euro Cells and Mater, 2002, 3(2):7-8; Turnbull, D. H. et al. Proc. Intl Soc Mag Reson Med, 2001, 9:359; Dunning, M. D. et al. J Neurosci, 2004, 24(44):9799-810; and Kaufman, C. L. et al. Transplantation, 2003, 76(7):1043-1046).

The nanoparticles and/or cells can be administered to a subject in isolation or within a pharmaceutical composition comprising the cells and/or nanoparticles, and a pharmaceutically acceptable carrier. As used herein, a pharmaceutically acceptable carrier includes solvents, dispersion media, coatings, antibacterial and antifungal agents, isotonic agents, and the like. Pharmaceutical compositions can be formulated according to known methods for preparing pharmaceutically useful compositions.

The nanoparticles and/or can be administered on or within a variety of carriers that can be formulated as a solid, liquid, semi-solid, etc. For example, genetically modified cells or non-genetically modified cells can be suspended within an injectable hydrogel composition (U.S. Pat. No. 6,129,761) or encapsulated within microparticles (e.g., microcapsules) that are administered to the patient and, optionally, released at the target anatomical site (Read T. A. et al., Nature Biotechnology, 2001, 19:29-34, 2001; Joki T. et al., Nature Biotechnology, 2001, 19:35-38; Bergers G. and Hanahan D., Nature Biotechnology, 2001, 19:20-21; Dove A. Nature Biotechnology, 2002, 20:339-343; Sarkis R. Cell Transplantation, 2001, 10:601-607).

Carriers for delivery of cells and/or nanoparticles are preferably biocompatible and optionally biodegradable. Suitable carriers include controlled release systems wherein the cells and/or the biological factors produced by the cells are released from the carrier at the target anatomical site or sites in a controlled release fashion. The mechanism of release can include degradation of the carrier due to pH conditions, temperature, or endogenous or exogenous enzymes, for example.

As applicable, the nanoparticles and/or cells can be grown, cultured, stored, and/or administered in or on various scaffolds, such as synthetic or biological tissue scaffolds (Griffith G. and Naughton G., Science, 2002, 295:1009-1013; Langer R., Stem Cell Research News, Apr. 1, 2002, pp. 2-3). Porous scaffold constructs can be composed of a variety of natural and/or synthetic matrices, such as biominerals (e.g., calcium phosphate) and polymers (e.g., alginate) that are optionally cross-linked, and serve as a template for cell growth and proliferation, and ultimately tissue formation. Three-dimensional control of pore size and morphology, mechanical properties, degradation and resorption kinetics, and surface topography of the scaffold can be optimized for controlling cellular colonization rates and organization within an engineered scaffold/tissue construct. In this way, the morphology and properties of the scaffold can be engineered to provide control of the distribution of bioactive agents (e.g., proteins, peptides, etc.) and cells. In addition to use as vehicles for delivery of the cells and/or nanoparticles, scaffolds can be utilized to grow the cells in vitro, preferably in the presence of free radical scavenging nanoparticles.

Scaffolds can contain interconnecting networks of pores and facilitate attachment, proliferation, and biosynthesis of cartilaginous matrix components, where desired. For example, synthetic or biological scaffolds carrying bone cells, such as chondrocytes, of the subject invention can be administered to a patient in need thereof. Chitosan scaffolds, which are biocompatible and enzymatically degraded in vivo, can be seeded with chondrocytes proliferated according to the methods of the subject invention and implanted. An alginate scaffold can be fabricated in the shape of a heart valve, seeded with proliferated cells of the invention, and implanted within a patient in need thereof. Because alginate does not naturally provide anchorage points for cells, in order to facilitate cell attachment, the peptide sequence R-G-D (Arginine-Glycine-Aspartic acid) can be utilized to act as a ligand for cell integrins and can be linked to alginate.

The terms “comprising”, “consisting of” and “consisting essentially of” are defined according to their standard meaning. The terms may be substituted for one another throughout the instant application in order to attach the specific meaning associated with each term.

The terms “isolated” or “biologically pure” refer to material that is substantially or essentially free from components which normally accompany the material as it is found in its native state. Thus, isolated cells in accordance with the invention preferably do not contain materials normally associated with the cells in their in situ environment.

As used in this specification, the singular forms “a”, “an”, and “the” include plural reference unless the context clearly dictates otherwise. Thus, for example, a reference to “a cell” includes more than one such cell (e.g., can include tissue and organs) or type of cell. A reference to “a nanoparticle” includes more than one such nanoparticle, and so forth.

The practice of the present invention can employ, unless otherwise indicated, conventional techniques of molecular biology, microbiology, recombinant DNA technology, electrophysiology, and pharmacology that are within the skill of the art. Such techniques are explained fully in the literature (see, e.g., Sambrook, Fritsch & Maniatis, Molecular Cloning: A Laboratory Manual, Second Edition (1989); DNA Cloning, Vols. I and II (D. N. Glover Ed. 1985); Perbal, B., A Practical Guide to Molecular Cloning (1984); the series, Methods In Enzymology (S. Colowick and N. Kaplan Eds., Academic Press, Inc.); Transcription and Translation (Hames et al. Eds. 1984); Gene Transfer Vectors For Mammalian Cells (J. H. Miller et al. Eds. (1987) Cold Spring Harbor Laboratory, Cold Spring Harbor, N.Y.); Scopes, Protein Purification Principles and Practice (2nd ed., Springer-Verlag); and PCR: A Practical Approach (McPherson et al, Eds. (1991) IRL Press)), each of which are incorporated herein by reference in their entirety.

Following are examples which illustrate procedures for practicing the invention. These examples should not be construed as limiting. All percentages are by weight and all solvent mixture proportions are by volume unless otherwise noted.

### Example 1

### Synthesis of Cerium Oxide Nanoparticles

CeO₂ nanoparticles with a particle size of 2-3 nm have been synthesized previously using the reverse micelle method. However, the surfactant sodium bis(2-ethylhexyl) sulphosuccinate (AOT) was used (see, for example, J. Phys.: Condens. Matter, 2001, 13:5269-5283). To improve biocompatibility and reduce potential toxicity of residual surfactant, a bio-surfactant phosphatidylcholine (i.e., soy bean lecithin) was used to form reverse micelles during synthesis. Phosphatidylcholine naturally occurs in cell membranes. Therefore, phosphatidylcholine for reverse micelle formation is expected to reduce the impact of residual surfactants to cell cultures and transplants and the patient. However, large amounts of lecithin have proven detrimental to the success and therefore more than 90% is removed in washing steps. The washing removes both toluene and the lecithin. Furthermore, the as synthesized nanoparticles require colloidal stabilization after removing the first surfactant in the washing step. Therefore, a specifically adsorbing compound was introduced into the system that cannot be washed out and is able to induce colloidal stabilization in aqueous systems like cell culture media, serum or blood. This compound is tri sodium citrate, i.e., the sodium salt of citric acid. Citric acid is fully biocompatible and part of the Krebs cycle in the cell. Therefore, nanocomposite particles comprising a coating of citrate around ceria are expected to be more biocompatible than ceria alone. These novel nanocomposite particles showed superior reduction of free radicals in chemical tests as well as in cell cultures and inside cells compared to commercially available ceria nanoparticles.

These represent new nanoparticles and a new method for their fabrication. Distinctions from prior nanoparticles include, for example, the two surfactants lecithin and trisodium citrate (citric acid can also be used, adjusting the pH, or the monosodium citrate or di sodium citrate can be used). Any other salts of citrate can also be used (e.g., ammonium salt, etc.).

Experimental Procedure

Phosphatidylcholine (laboratory grade), toluene (laboratory grade), cerium (III) nitrate hexahydrate (99.5%, M.W.=434.22 g/mole), and ammonium hydroxide (NH₃ content 28˜30%) were purchased from Fisher Scientific and used without further purification. 2.285 g of phosphatidylcholine were dissolved in 100 ml of toluene, forming reverse micelles in the nonpolar medium. Next, 5 ml of 0.1M cerium nitrate aqueous solution was pipetted into the yellowish medium. The system was strongly stirred for 30 minutes to achieve evenly distributed reverse micelles loaded with precursor solution forming a nanoreactor inside the phosphatidylcholine. Next, the pH of the nanoreactor was increased by addition of 10 ml of 1.5M ammonium hydroxide solution into the flask. After 45 minutes of strong stirring, crystalline CeO₂ nanoparticles precipitated inside the reverse micelles. These nanoparticles were centrifuged using 10,000 rpm. The sediment (the nanoparticles coated with lecithin in presence of residual toluene and water) was washed with 50 ml methanol, 50 ml ethanol, and 50 ml water, respectively and centrifuged at 10,000 rpm after each wash. The purified nanocrystalline ceria nanoparticles were then treated with 100 ml of 1.5M sodium citrate solution for 48 hours with ultrasound for redispersion. The appearance of the nanoparticle suspension changed from turbid to transparent indicating successful dispersion of the coated nanocomposite particles. The suspension was then filtered through 200 nm syringe filter for sterilization.

### Example 2

### Cellular Uptake of Cerium Oxide Nanoparticles and Reduction of Oxidative Stress

The inventors have developed several types of nanoparticles, nanoparticle coatings for cell uptake, a method for making these nanoparticles, and have tested them successfully with human islets of Langerhans as well as other mammalian cell lines.

The present invention pertains to the application of these nanoparticles to diminish oxidative stresses by catalyzing free radicals. This process can take place both intracellularly, and thus benefit the viability and function of the cells, and in solution and thus can be used for a wide variety of industrial applications.

The data disclosed herein clearly show a reduction in intracellular free radical concentration when these nanoparticles were provided as a supplement to standard culture media.

The nanoparticles were cerium oxide or cerium oxide-based materials, and are under 20 nm in size. These nanoparticles are well dispersed in solution, forming stabilized nanoparticle suspensions. The present inventors have shown that the uptake rates of cerium oxide nanoparticles by cells are consistent with the concentration of nanoparticles in the suspension. Also, the reduction of free radical concentration is consistent with the increasing concentration of nanoparticle suspensions.

The present invention provides novel systems that further improve the capability of reducing oxidative stresses in the cells. The capability of diminishing oxidative stresses by cerium oxide or other free radical scavenging nanoparticles can be enhanced by increasing oxygen vacancies, increasing crystallinity, decreasing particle size, and increasing surface area of these nanoparticles; each of which can be achieved by pre-reducing the nanoparticles or doping/mixing other elements/oxides into the nanoparticles.

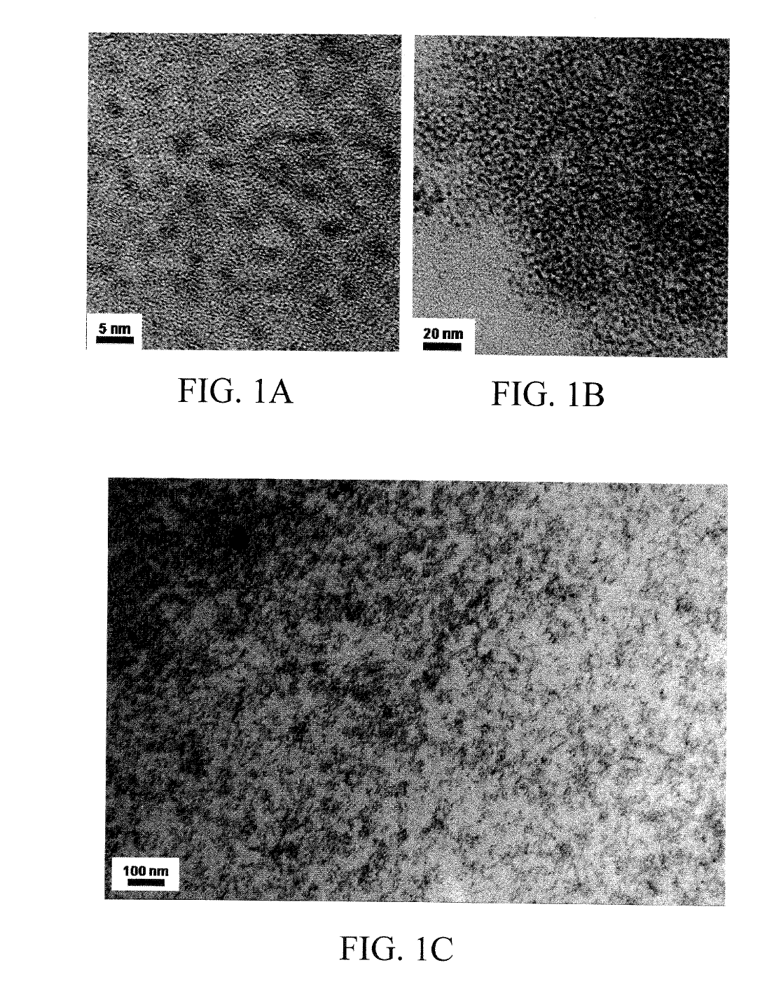

FIGS. 1A-1C show TEM of cerium oxide nanoparticles. The nanoparticles are 3 nm to 5 nm in size (FIG. 1A). They are weakly flocculated after synthesis (FIG. 1B), and can be dispersed in solution (FIG. 1C).

The nanoparticles that have been synthesized have a particle size smaller than 20 nm and can undergo uptake by cells. Since the present inventors have shown that the presence of ceria nanoparticles can decrease the concentration of free radicals in cells and they are non-toxic to cells, the nanoparticles can perform free radical scavenging better than naturally occurring enzymes.

Of the many challenges facing the field of islet cell transplantation, one of the principle concerns has been that of islet viability in vivo. The identification of methods that enhance islet survival post transplantation is crucial for improving therapeutic outcomes. The free radical scavenging nanoparticles and methods of the invention can be used to prolong islet viability and improve islet function. While the specific mechanisms underlying these physiological enhancements remain unclear, the incorporation of semiconductor nanoparticles such as ceria may, like vitamin C and/or Vitamin E, prevent oxidative stresses.

Experimental Aims

1. Perform synthesis of ceramic nanoparticles, specifically CeO₂, with variation in particle size and Ce³⁺/Ce⁴⁺ ratio.

2. Evaluate the in vitro effects of these nanoparticles on murine islet viability and function.

The object of this project is to increase islet viability by incorporation of semiconducting nanoparticles. Semiconductor nanosized particles possess unique structural and physical properties as a result of their size controlled electronic band structure and increased interfacial area. The surface chemistry of these nanoparticles has been reported to impede oxidants and free radicals from production and propagation, since these grains encompass a preferential radical scavenging characteristic that is enhanced if more than one stable redox state of their constituent ions is available [5]. Although other chemicals, such as Heme Oxygenase-1, SOD mimetics, vitamin C and/or E have been shown to improve oxidative stress in transplanted islets, they degrade over a relatively short time span once being incorporated into the islet [6,7]. The radical scavenging process of nanoparticles is hypothesized to be based on the redox behavior of ceramic lattice cations switching their oxidation state, e.g., from Ce³⁺ to Ce⁴⁺. The radical scavenging has also been observed for Se nanoparticles and some fullerenes [3,8]. From a materials science perspective, multiple forms of additional nanoparticles could be tested depending on their electronic structures (bandgaps) and surface features. Previous work has been performed monitoring the effect of ceria nanoparticle administration on the longevity of organotypic rat brain cortical cultures.

The present inventors have synthesized and characterized various nanoparticles with band gaps from 0 to 4 eV. FIGS. 1A-1C demonstrate non-agglomerated ceria nanoparticles with a bandgap of 3 eV. Additionally, the inventors have generated CeO₂ nanoparticles with a core size of 4-5 nm and coated with either lecithin or citrate to facilitate their solution in aqueous media.

The first objective of these in vitro studies was to assess whether ceria nanoparticles were uptaken by insulin secreting cells and where they were deposited. To achieve this objective, murine insulinoma βTC-tet cells were exposed for 22 hours to culture media containing 2%, v/v suspension of CeO₂ nanoparticles. At the end of this exposure period, the cells were fixed and examined by TEM. FIG. 2A is a TEM of several βTC-tet cells obtained at 2,000×; FIG. 2B is 25,000× magnification of one of these cells. Ceria nanoparticles are identified by their electron rich dark spots and they are located in cytoplasmic lysosomes. As points of reference, the nucleus and secretory vesicles are also identified in the figures.

The intracellular concentration of cerium in βTC-tet cells was determined by exposing for 18 hrs to media containing CeO₂ nanoparticles at a concentration of either 100 or 1,000 nM. Following the exposure to ceria the cells were trypsinized, pelleted and digested in concentrated sulfuric acid. The acid digest was analyzed by Inductively Coupled Plasma (ICP) to determine the concentration of Cerium in the cells. Cells exposed to media containing 100 nM CeO₂ were shown to contain 0.016 pg/cell, while cells exposed to media containing 1,000 nM CeO₂ were shown to contain 0.029±0.009 pg/cell. By comparison, cells exposed to media that did not contain CeO₂ nanoparticles were shown to have an intracellular cerium concentration of 0.00037 pg/cell (˜50× less than cells exposed to media with 100 nM CeO₂).

The second objective was to assess whether ceria nanoparticles were detrimental to the metabolic and secretory activity of the cells. To achieve this objective, βTC-tet cells were exposed for 22 hours to culture media containing 2% v/v suspension of CeO₂ nanoparticles. Metabolic activity was assessed by measuring the rate of glucose consumption (GCR) over the 22 hours of exposure. Secretory activity was assessed by measuring glucose stimulated insulin secretion following the nanoparticle exposure using the protocol reported by Oca-Cossio et al. [9].

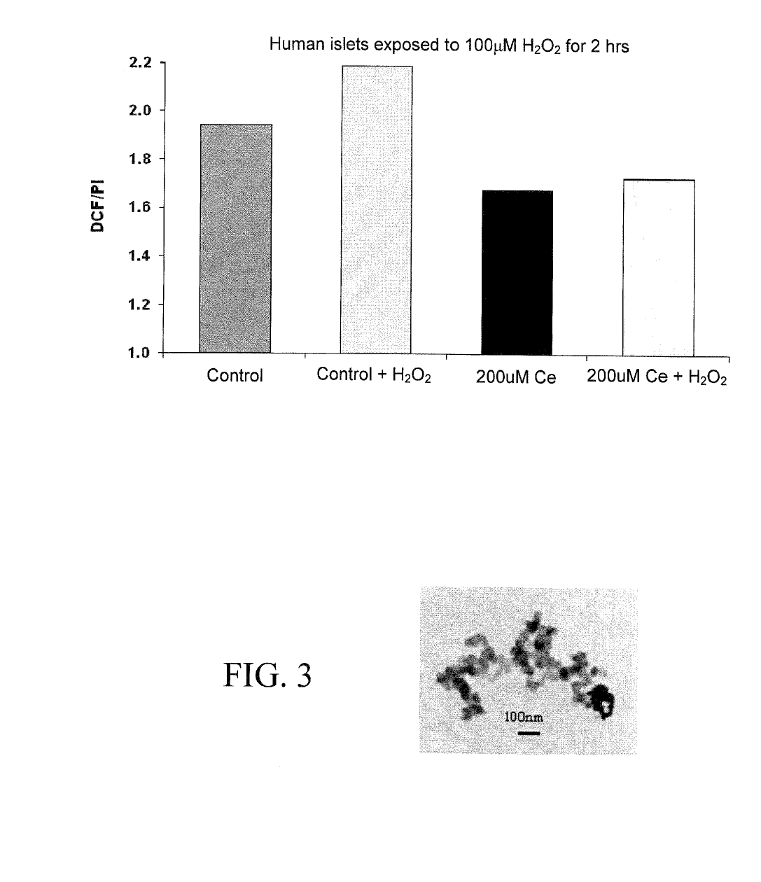

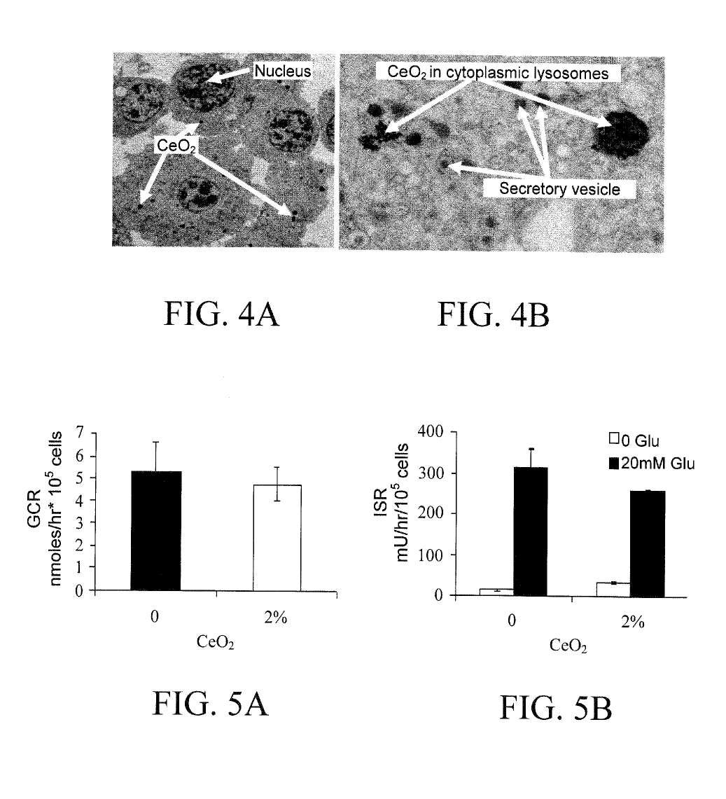

FIGS. 3A and 3B are bar graphs depicting the average GCR by βTC-tet cells over 22 hours (FIG. 3A) and the insulin secretion rates (ISR) by βTC-tet cells at 0 mM and 20 mM glucose (FIG. 3B). The data show that exposure to the nanoparticles did not have a statistically significant effect on GCR. It is important to note that these measurements were performed after the 22 hour ceria exposure was completed and that the cells were exposed to each glucose concentration for 20 minutes. As previously with the GCR measurements, the data show that exposure to ceria did not have a statistically significant effect on insulin secretion. These data demonstrate that ceria are uptaken by the cells and that they are not detrimental to either the metabolic or secretory activity of the cells.

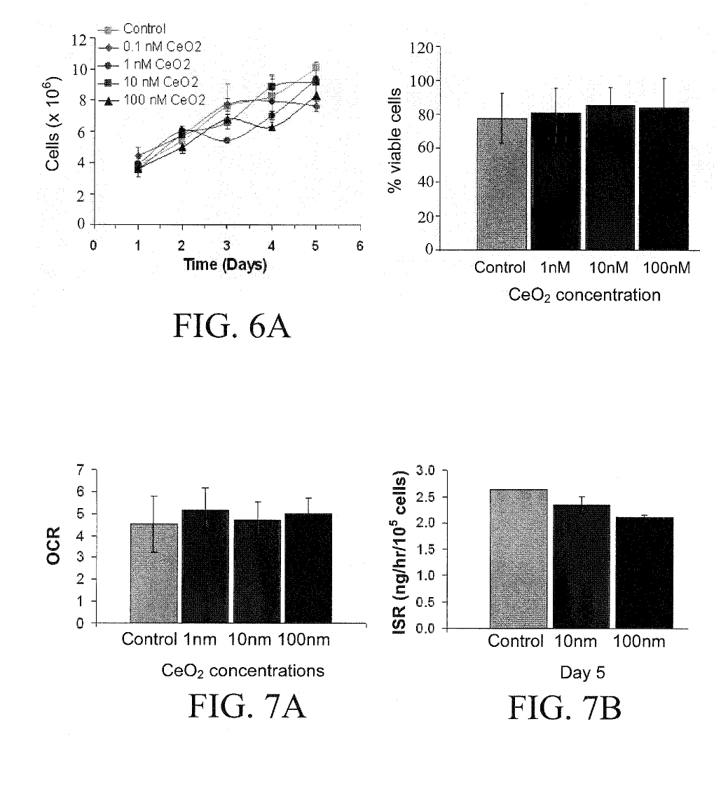

To assess the effect on cell growth, monolayer cultures of (βTC-tet cells were allowed to grow to confluence on T-25 flasks with DMEM culture media containing lecithin-coated CeO₂ nanoparticles at concentrations varying from 0.1 to 100 nM. Freshly trypsinized cells were allowed to attach on new flasks for 24 hours and then were fed with media containing CeO₂ nanoparticles at varying concentrations. FIG. 6A depicts temporal changes in the number of βTC-tet cells as a function of CeO₂ concentration in the culture media. The data suggest that a range in CeO₂ concentration from 0.1-100 nM did not affect the growth of βTC-tet cells. Furthermore, the percent of viable cells after exposure to CeO₂ containing media for 4 days was the same among the various cultures (FIG. 6B). The error bars, on either graph of FIG. 6A or 6B, depict the standard deviation of the mean based on triplicate runs. Metabolic activity of the cells was assessed by the rates of oxygen consumption (OCR) and insulin secretion (ISR) after 4 days of exposure to CeO₂ containing media (5^{th} day of culture). Both measurements were conducted with media containing 15 mM glucose. FIGS. 7A and 7B illustrate that there is no statistical significant effect on either oxygen consumption (FIG. 7A) or insulin secretion (FIG. 7B) following exposure to CeO₂ containing media.

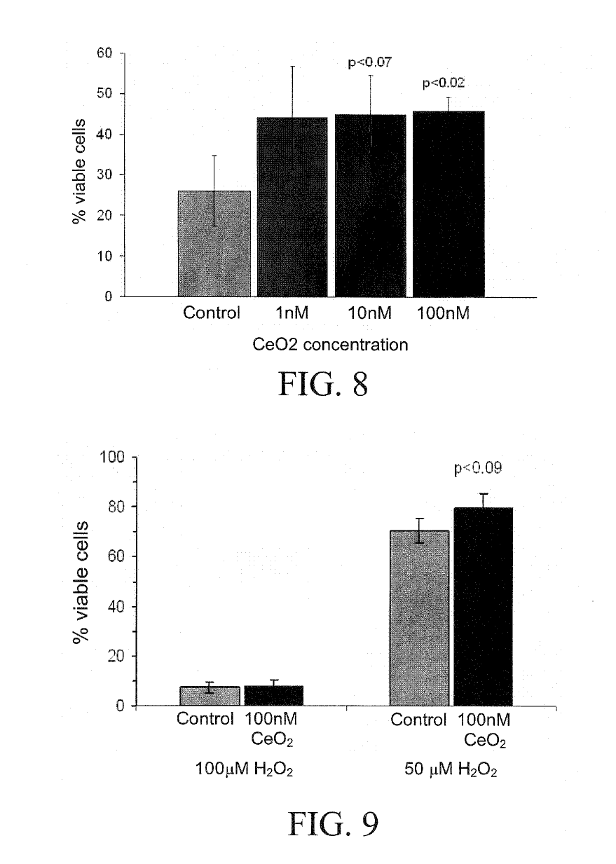

Experiments were conducted to asses the protective effect of CeO₂ nanoparticles against ROS generated by either serum deprivation or H₂O₂. For the serum deprivation study, freshly trypsinized βTC-tet cells were plated on new T-25 flasks and allowed to attach under standard culture conditions. Twenty four hours later, the cells were fed fresh fully supplemented media that contained lecithin-coated CeO₂ nanoparticles at a concentration of 0 (control), 1, 10 or 100 nM. Four days later, the flasks were confluent and the cells were fed with fresh media that were free of serum but supplemented with all other necessary ingredients. The cells were maintained under this serum-free condition for 4 additional days and the viability of the flasks was assessed by the trypan blue exclusion method at the end of the 4^{th} day. FIG. 8 shows the average percent of viable cells under each experimental condition. The data suggest that CeO₂ loaded cells are protected against ROS generated during serum deprivation. Although this protective effect does not appear to be concentration dependent (the average % viable cells is the same for cells exposed to 1, 10 or 100 nM CeO₂), only cells exposed to 100 nM CeO₂ benefited statistically (p<0.02) by the nanoparticles. Cells exposed to 10 nM approached statistical significance but did not achieve it (p<0.07). This is attributed to the high standard deviation generated by averaging the three independent measurements.

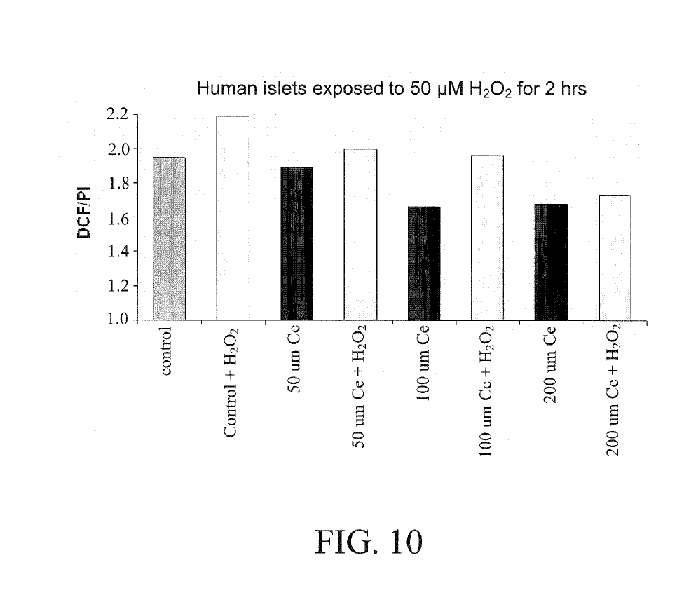

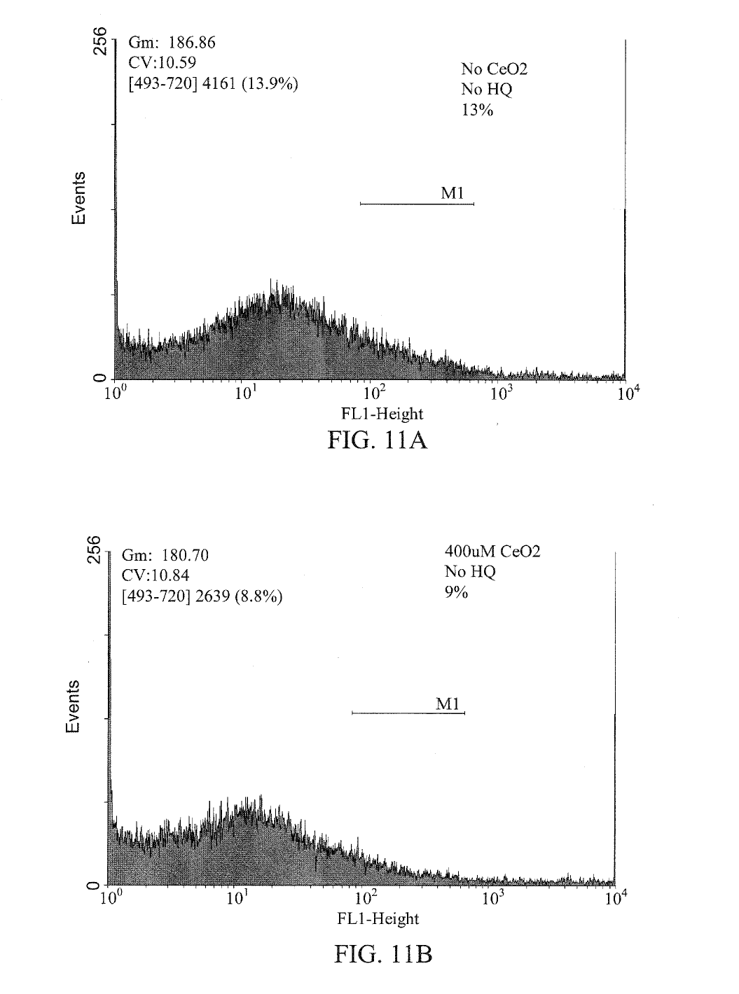

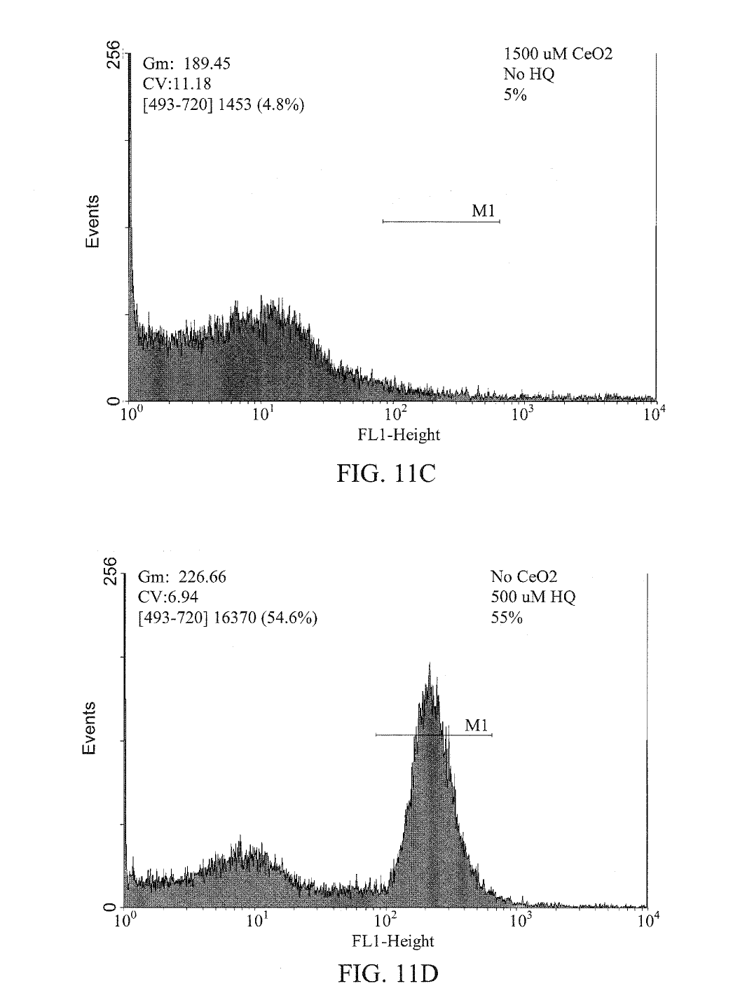

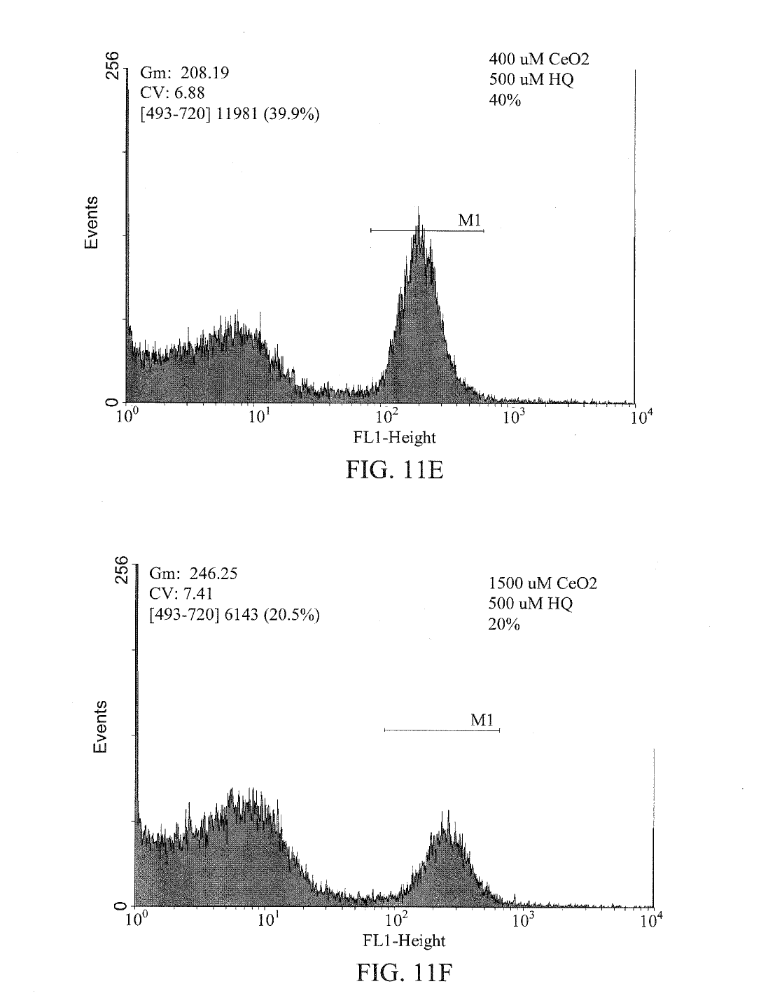

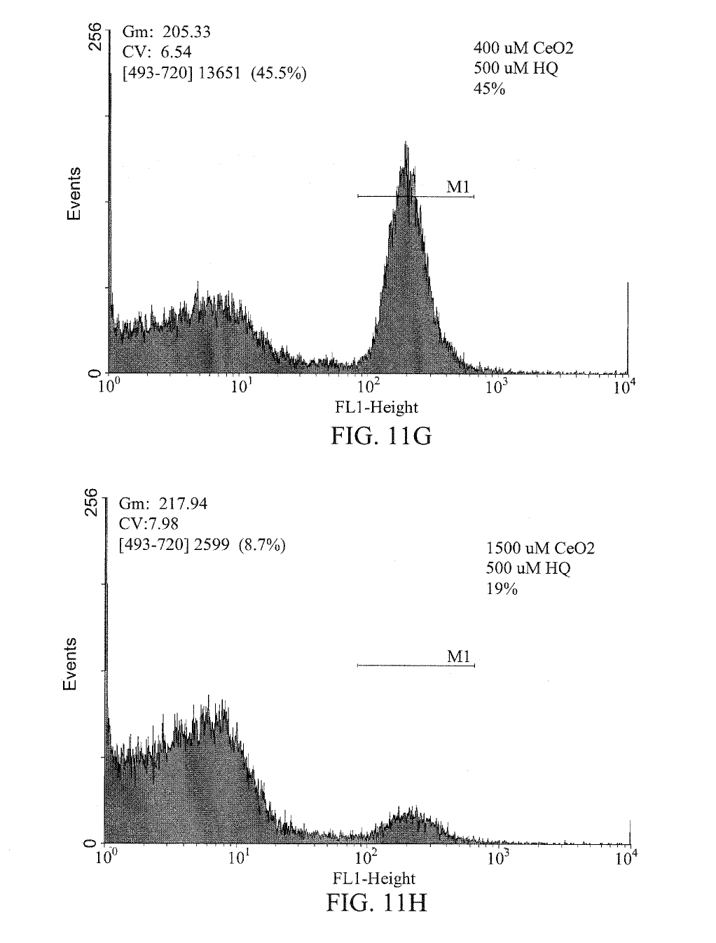

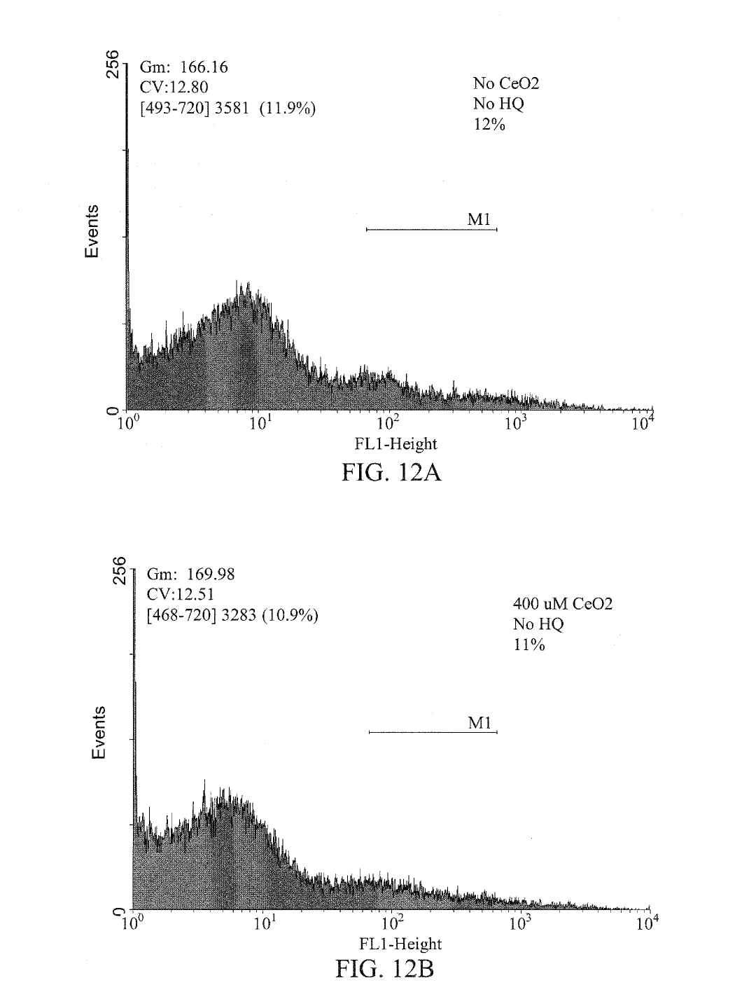

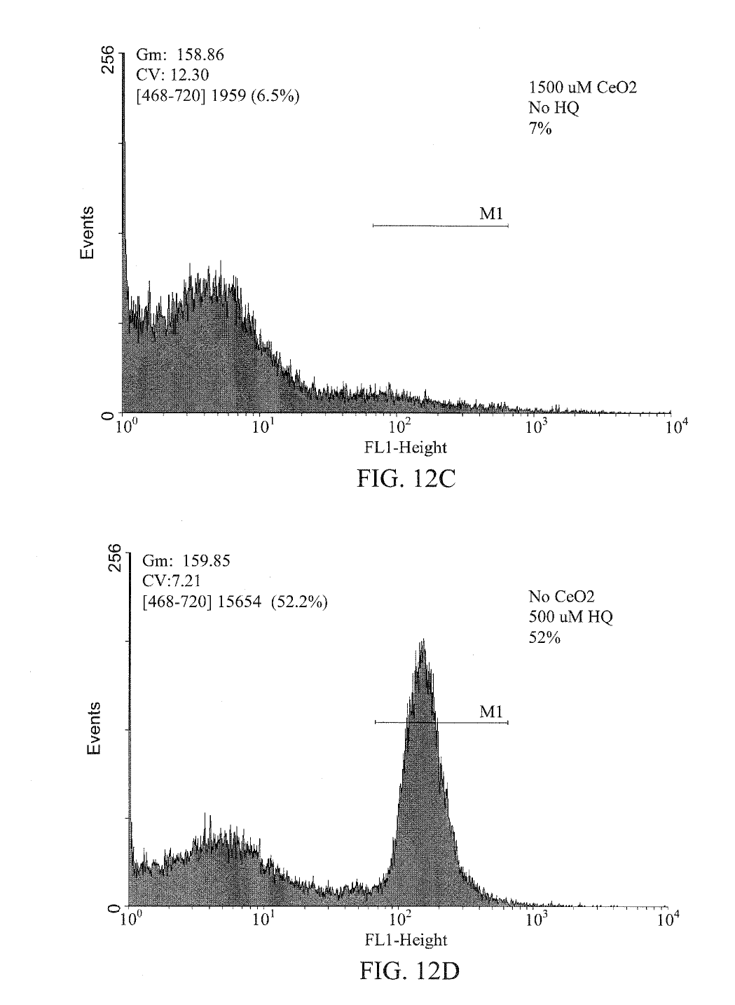

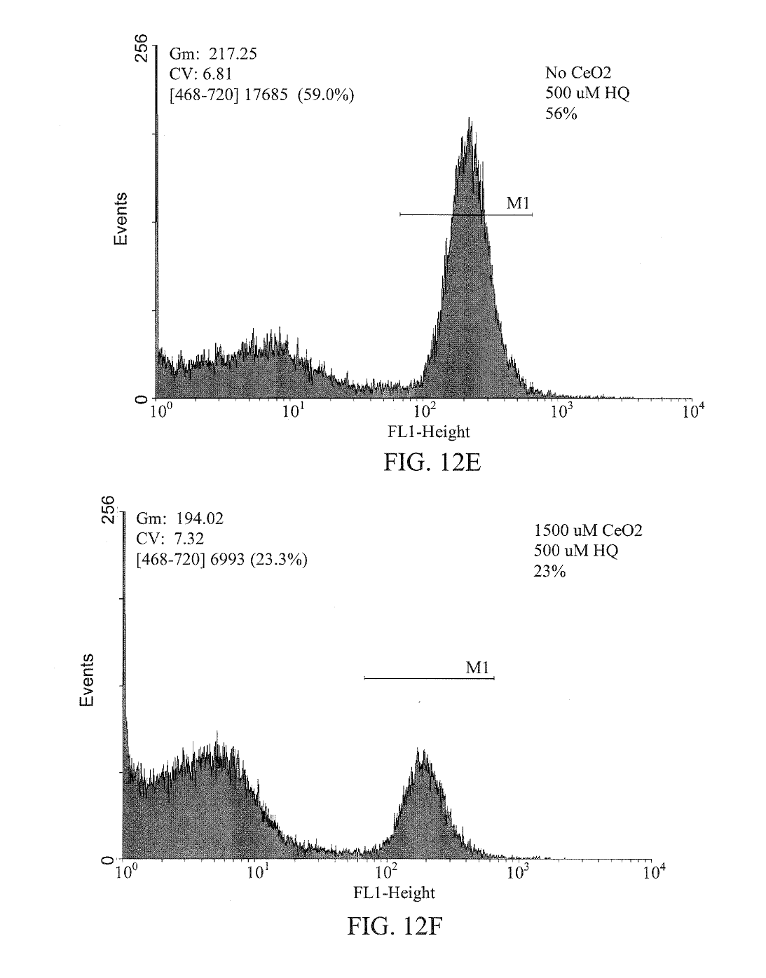

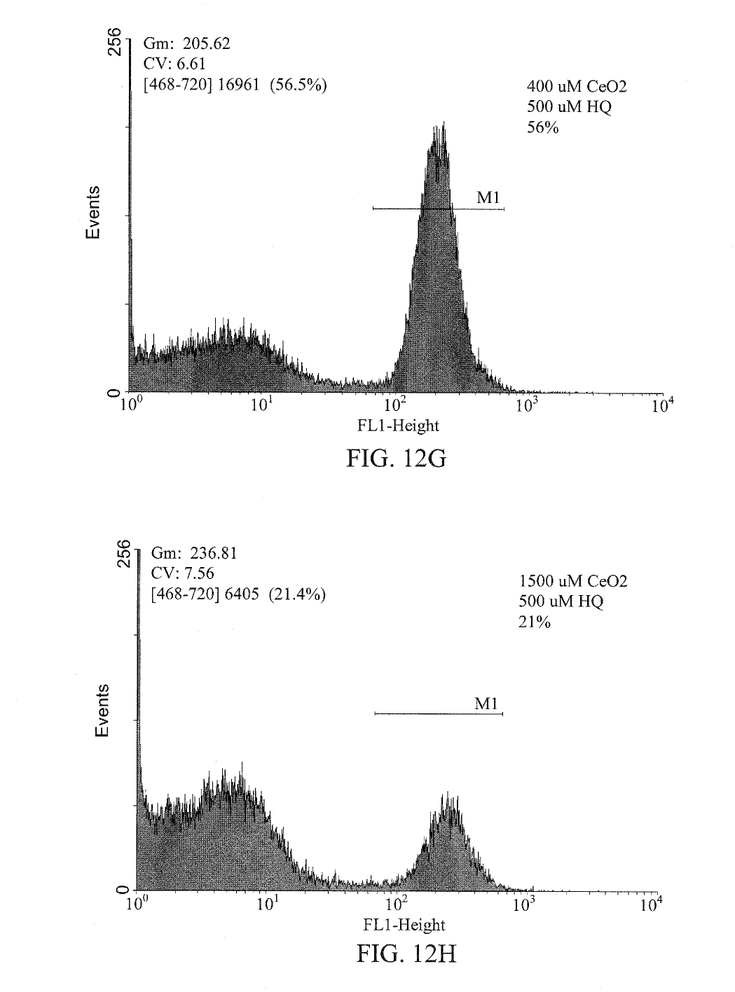

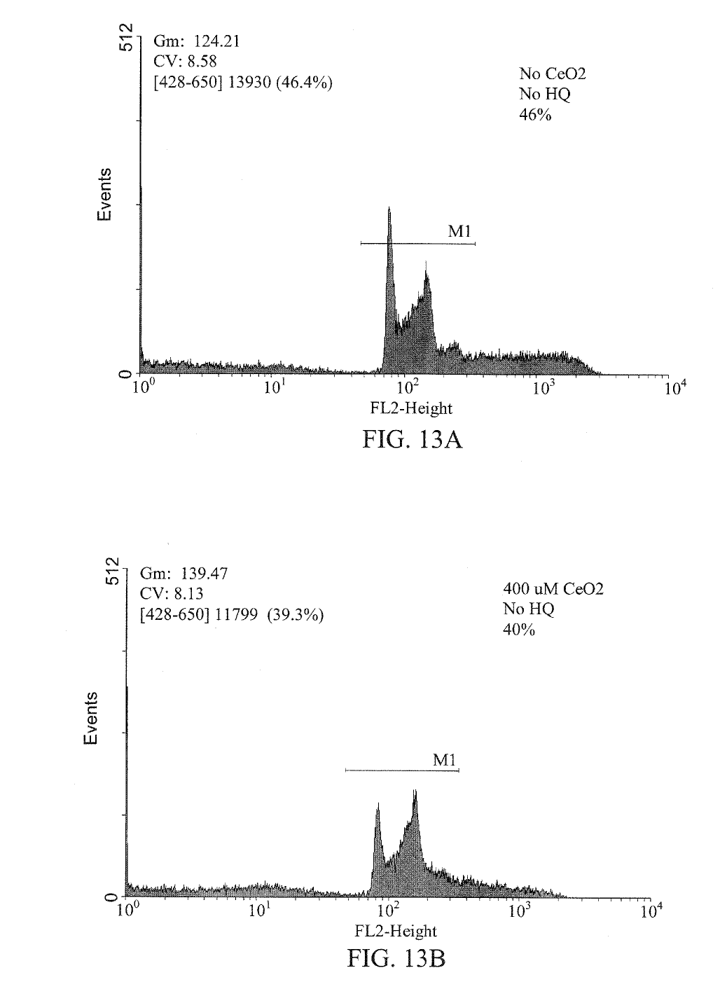

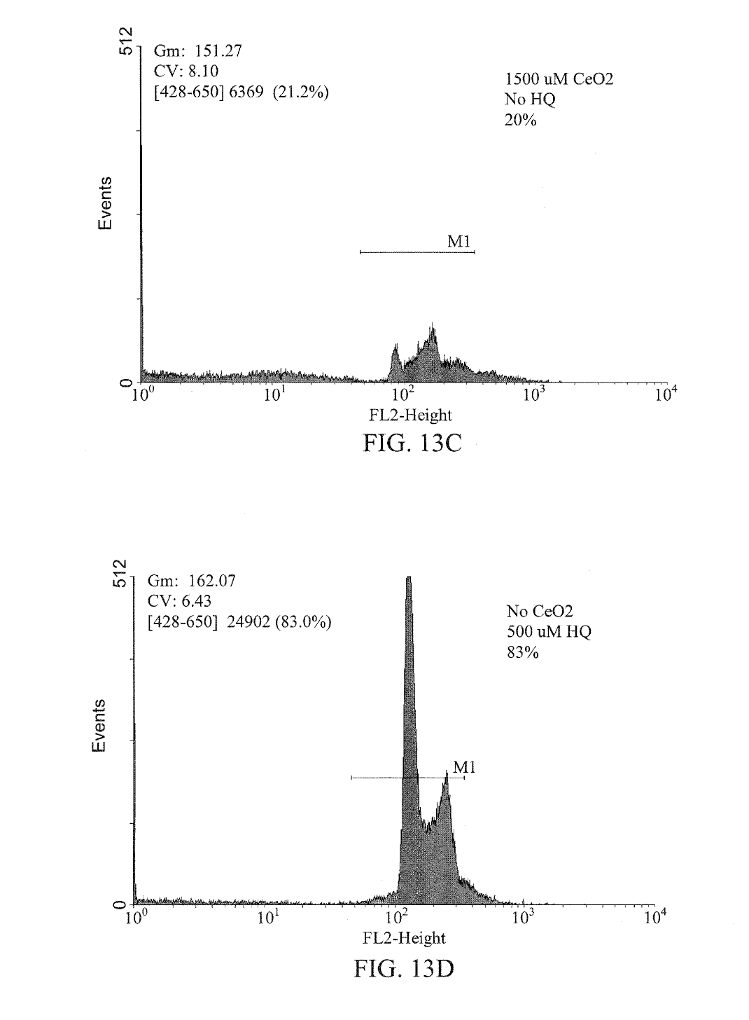

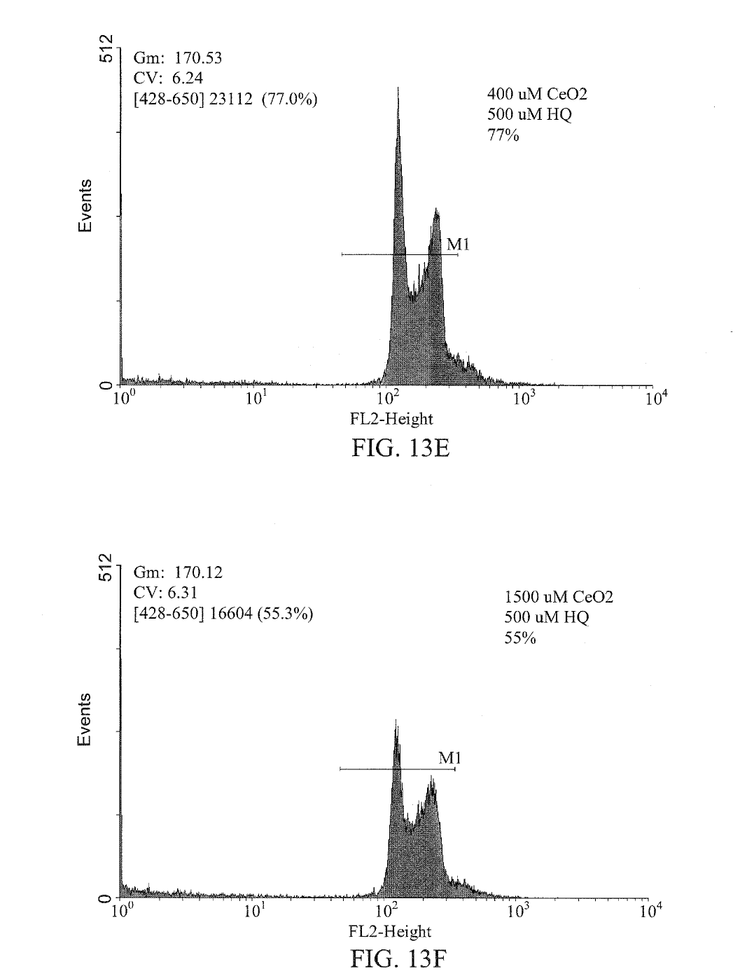

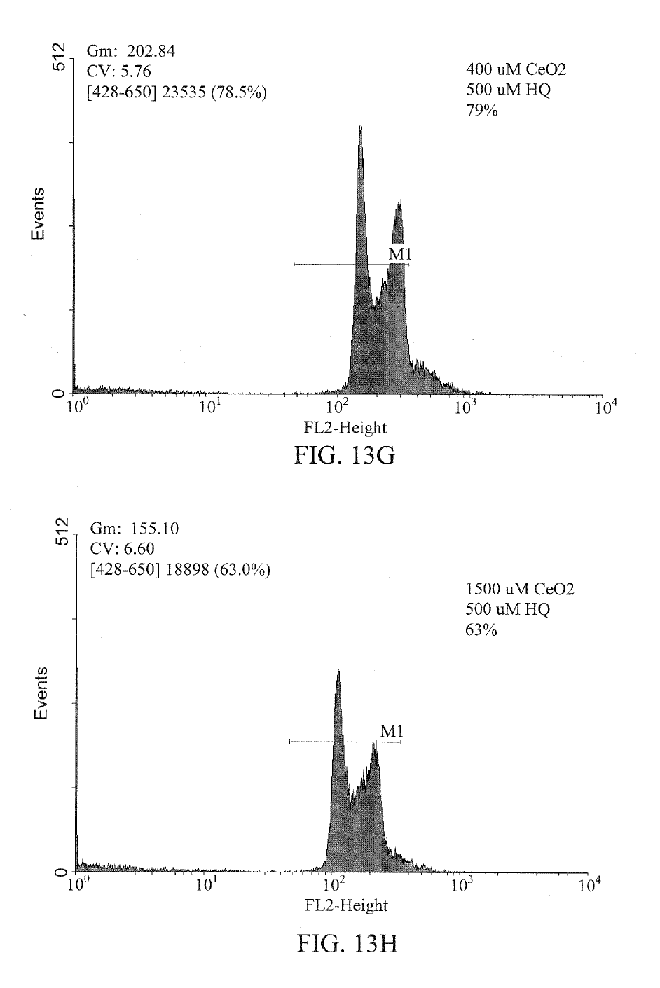

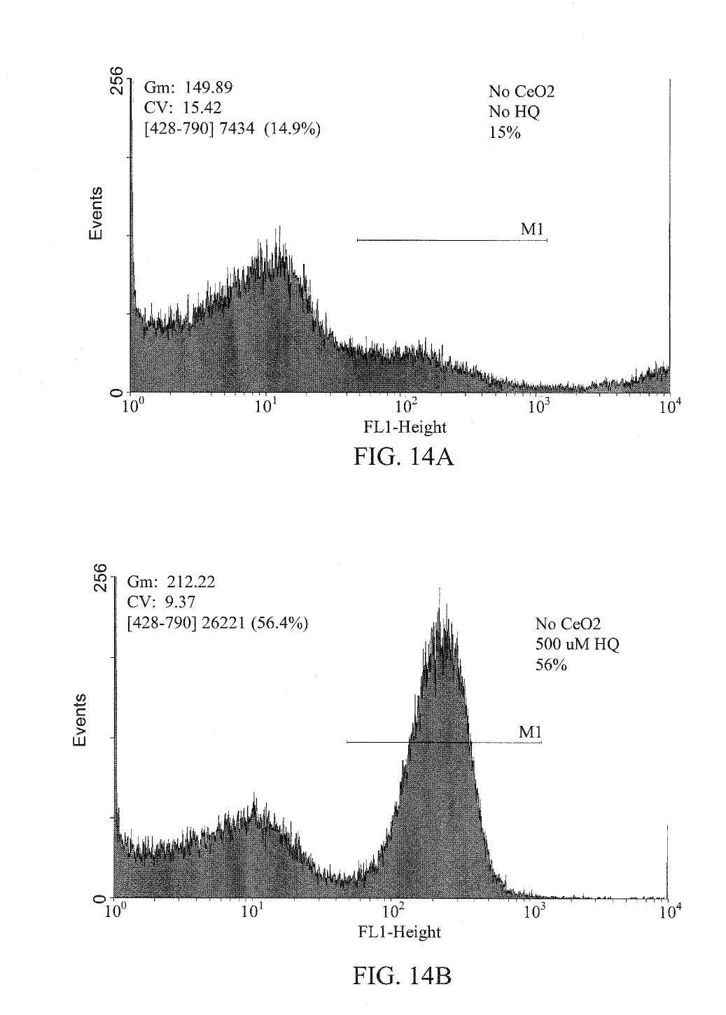

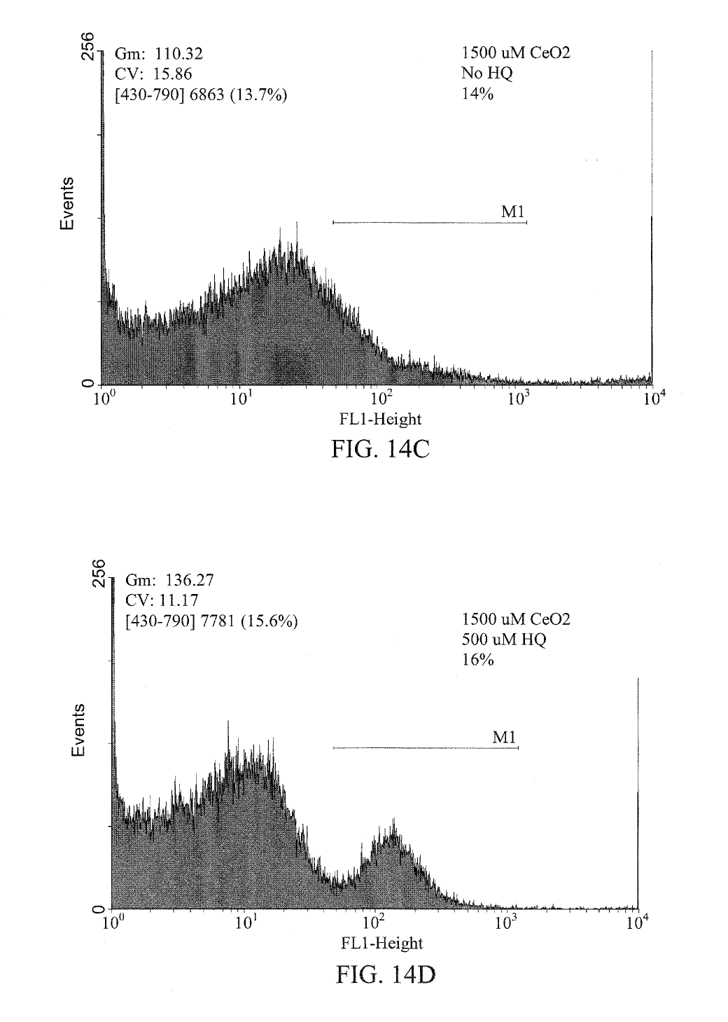

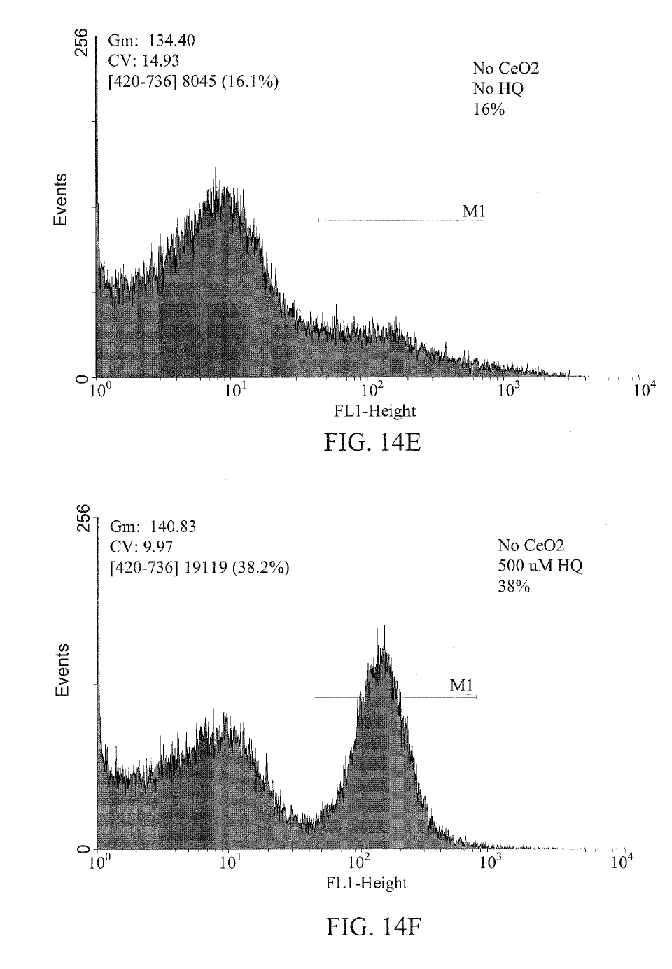

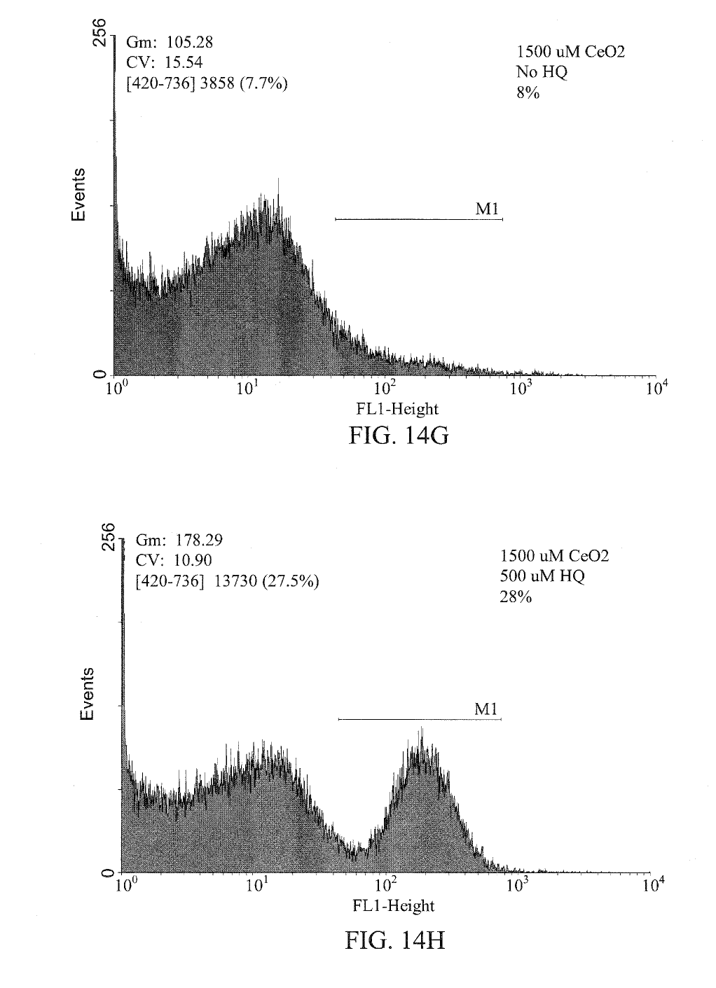

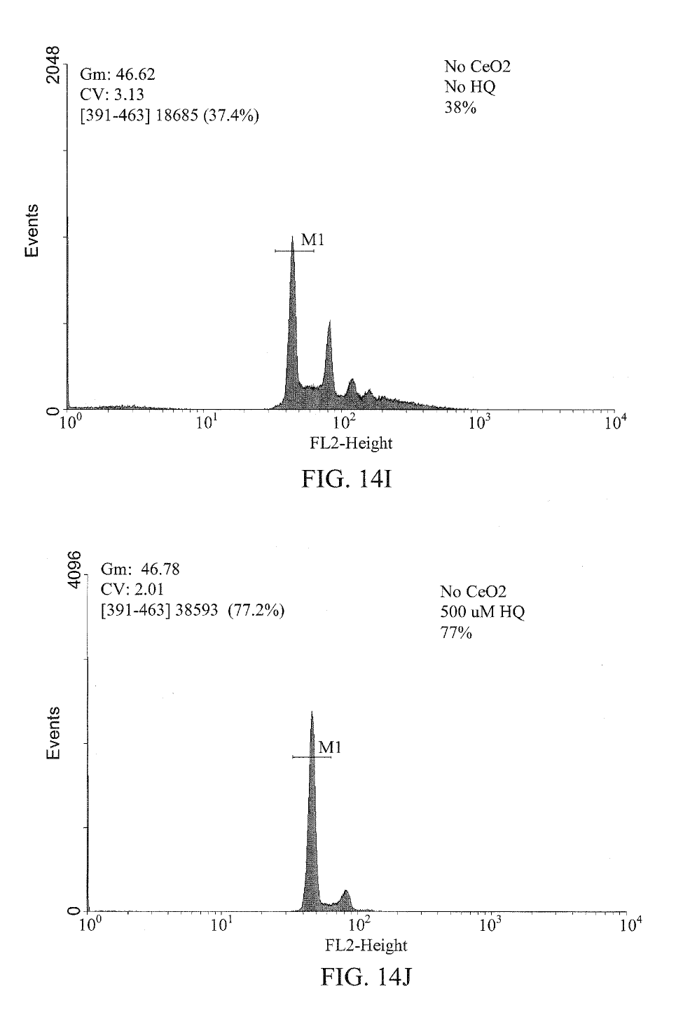

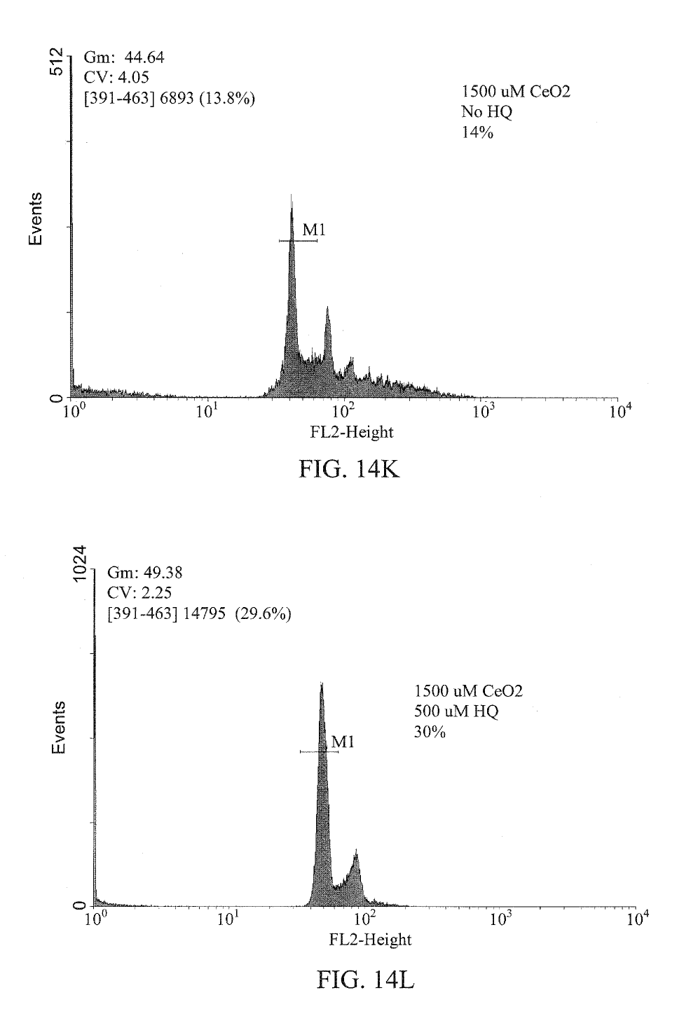

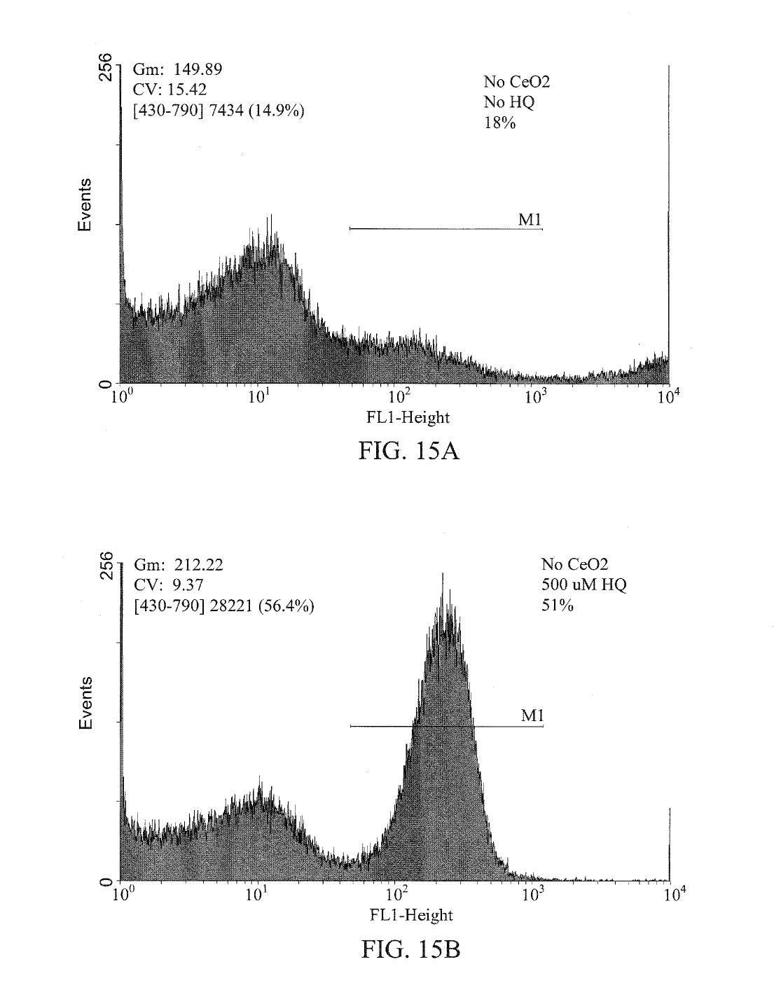

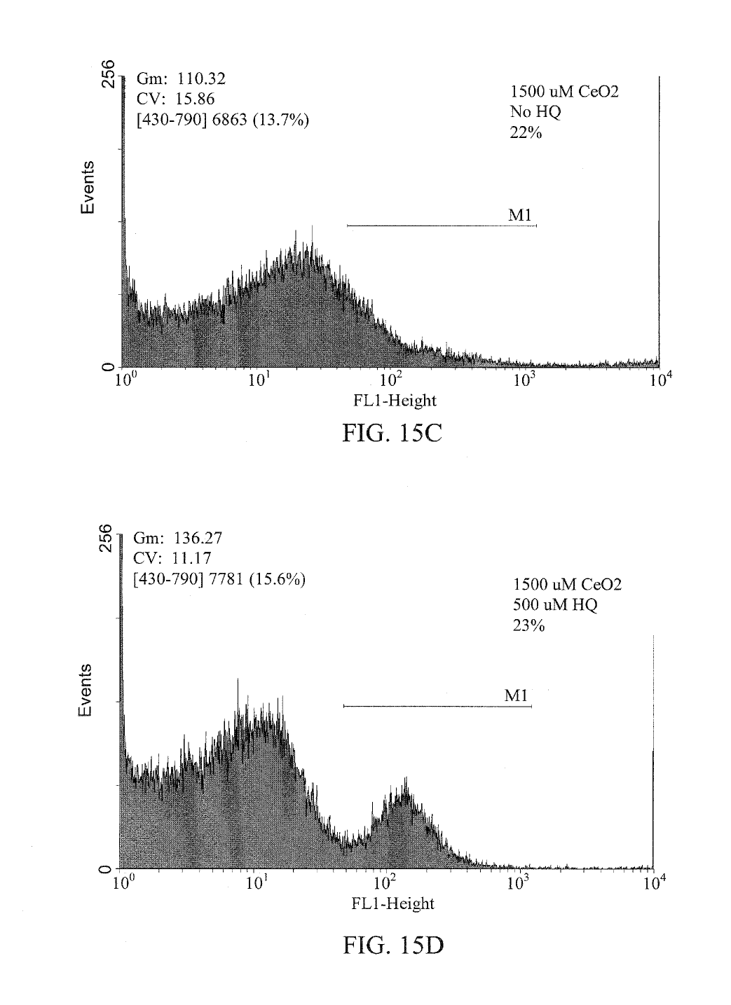

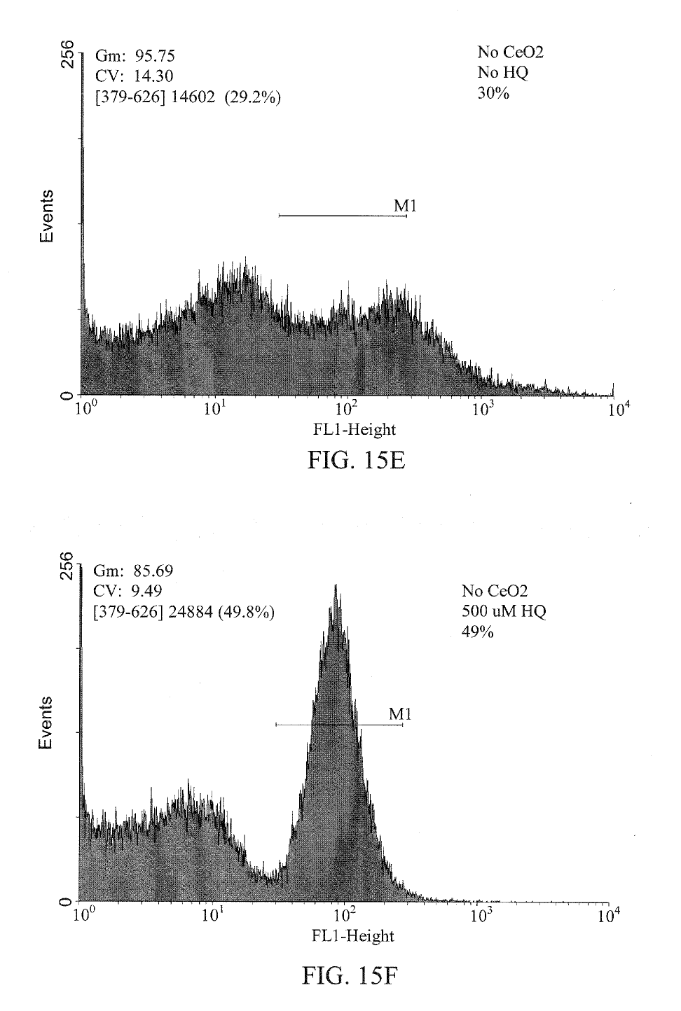

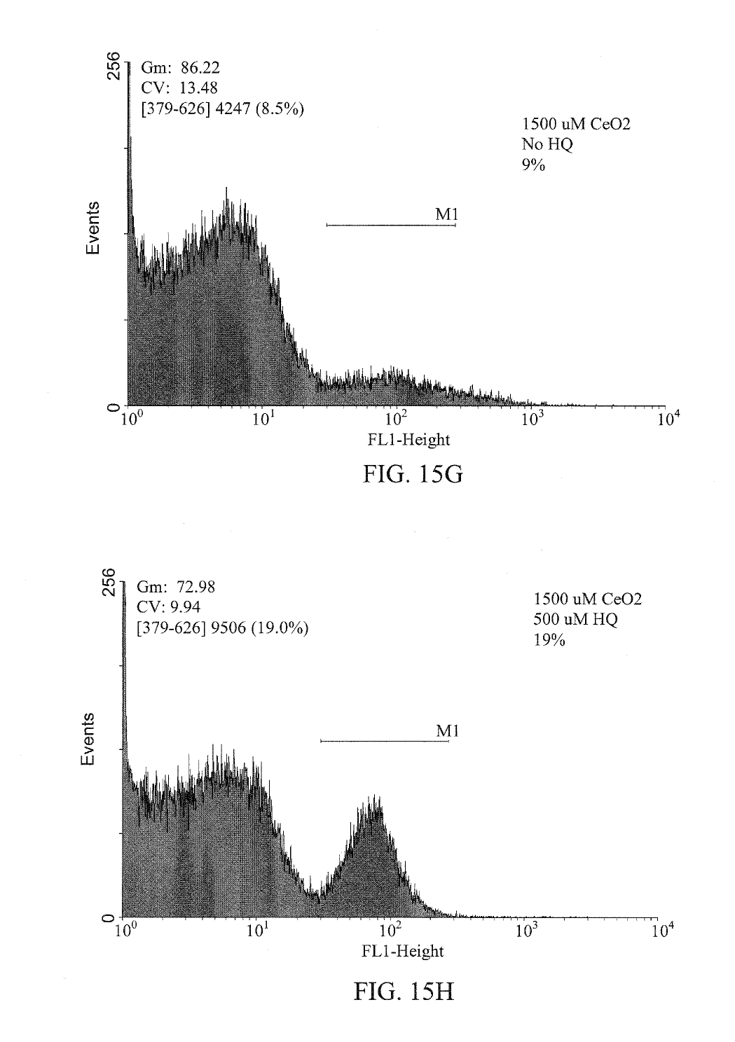

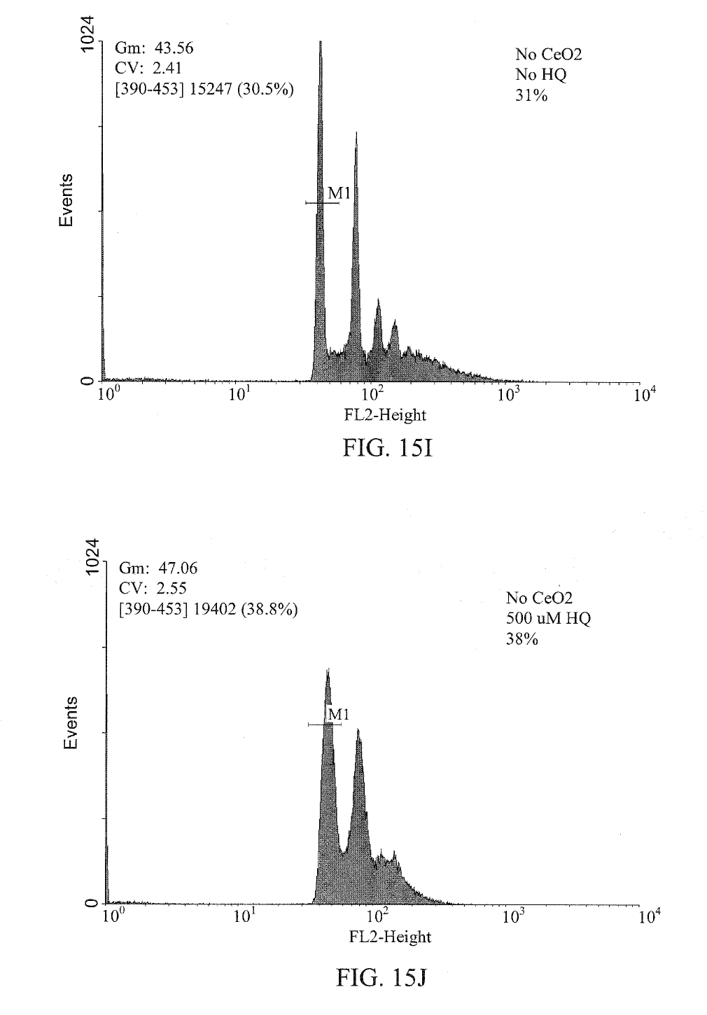

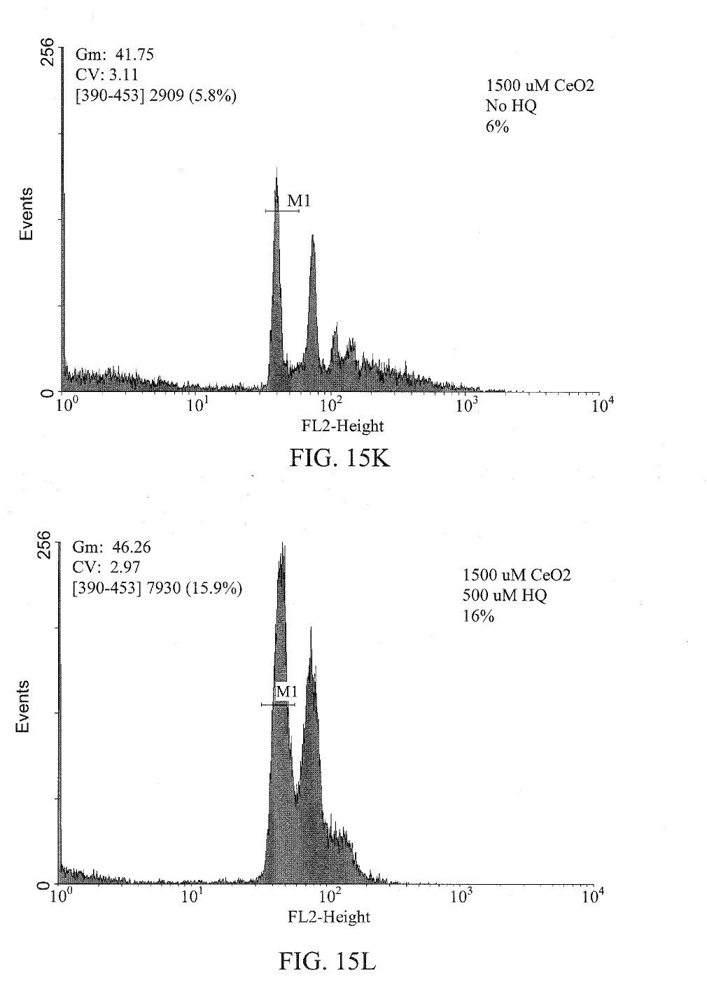

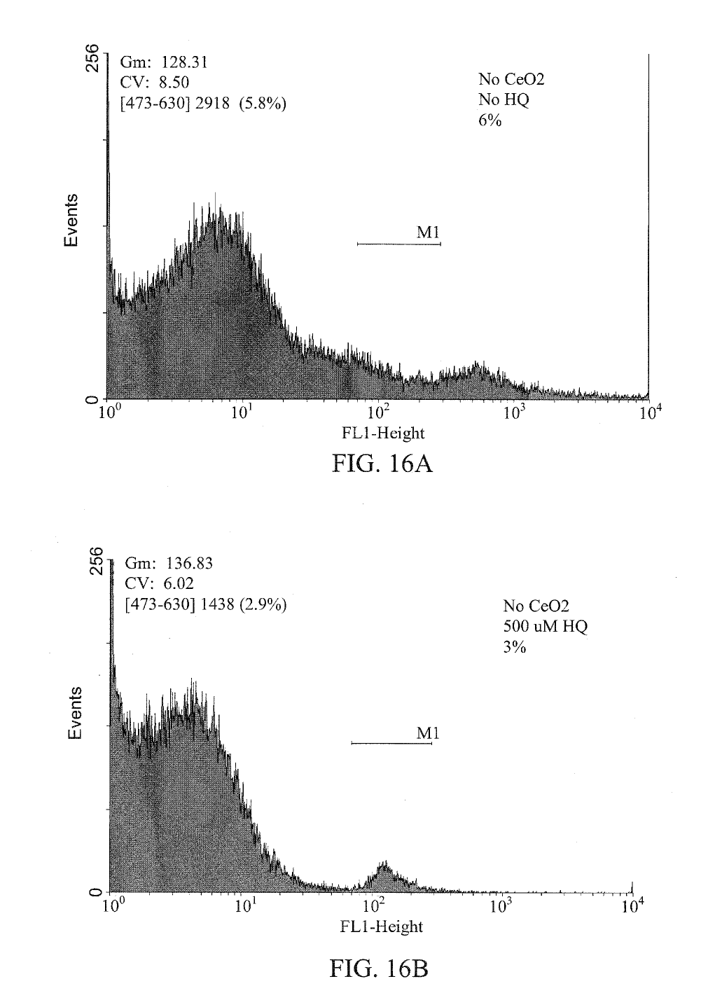

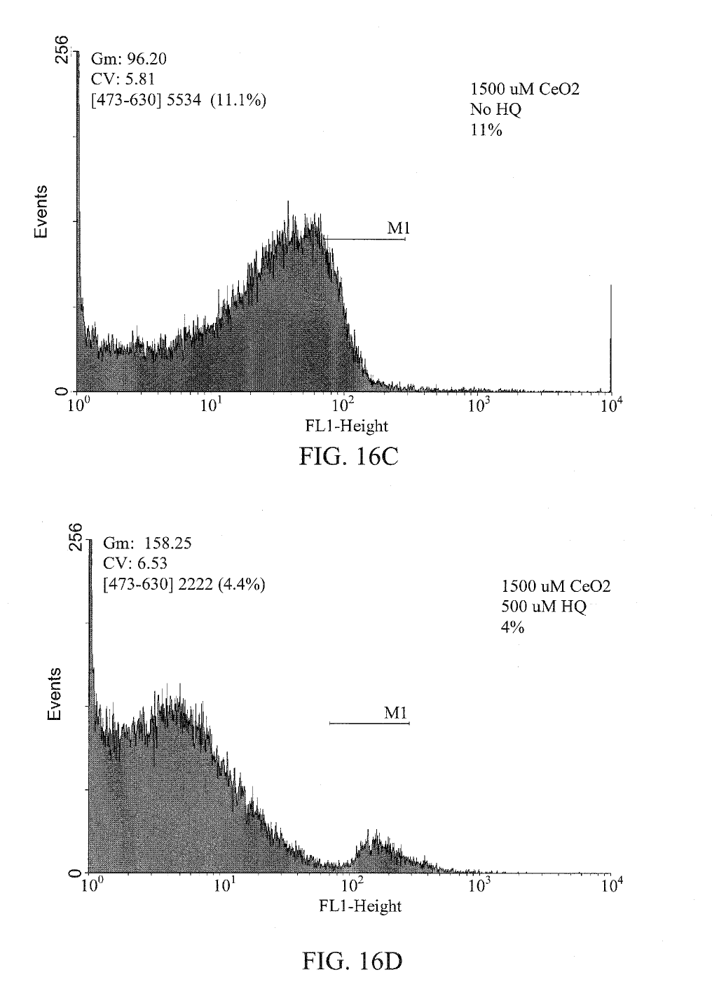

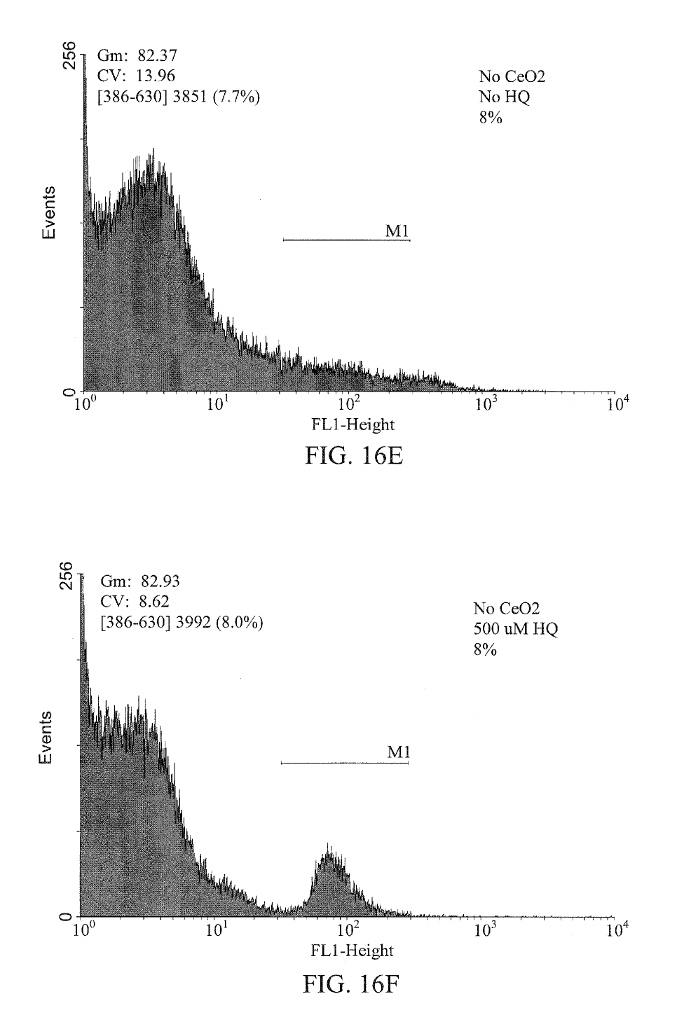

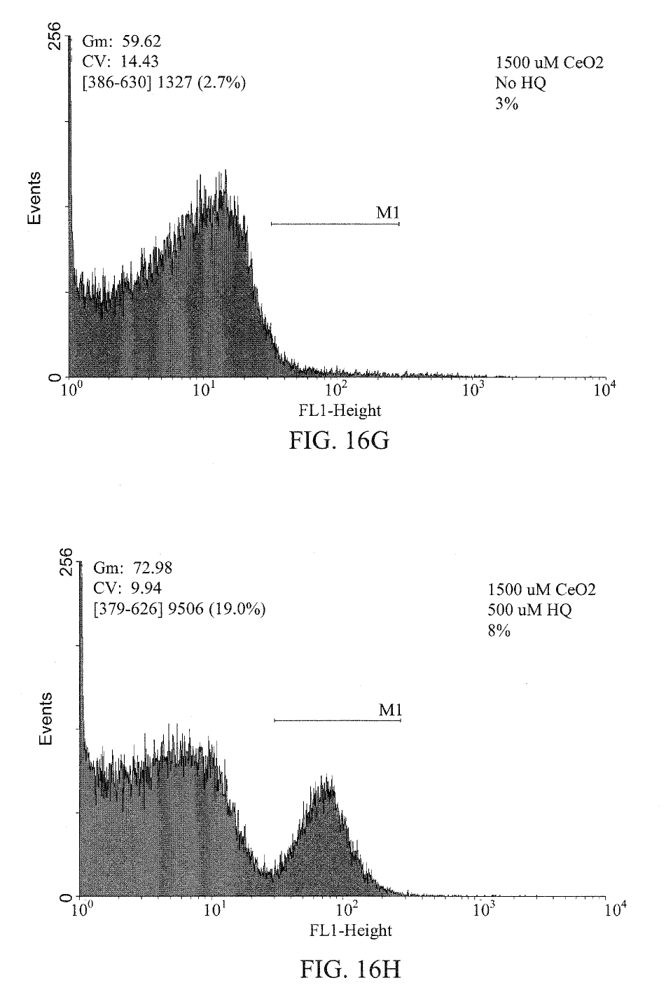

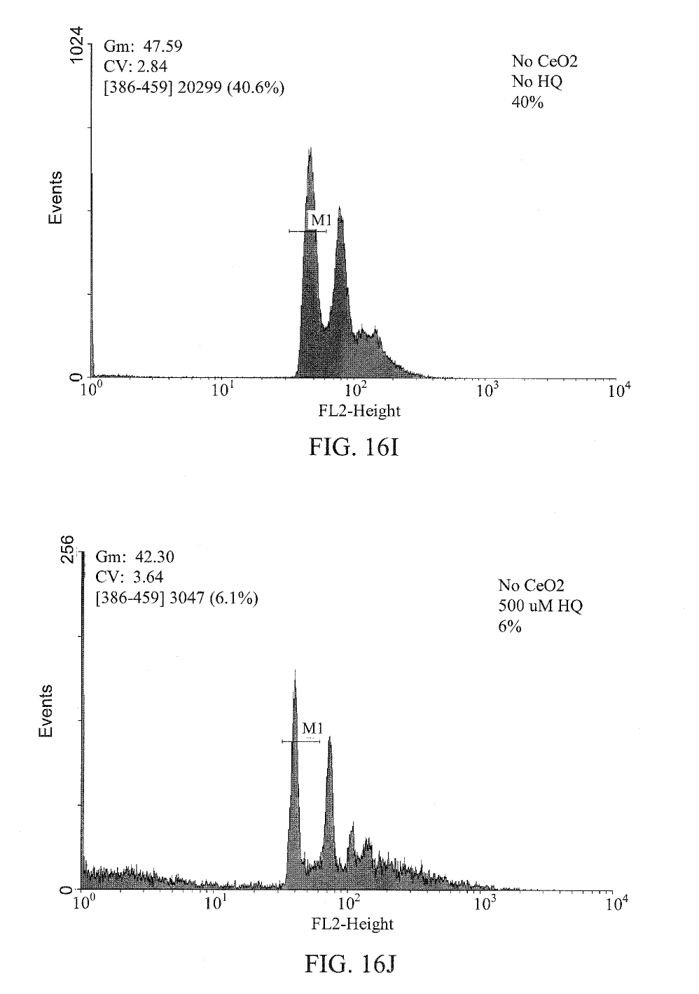

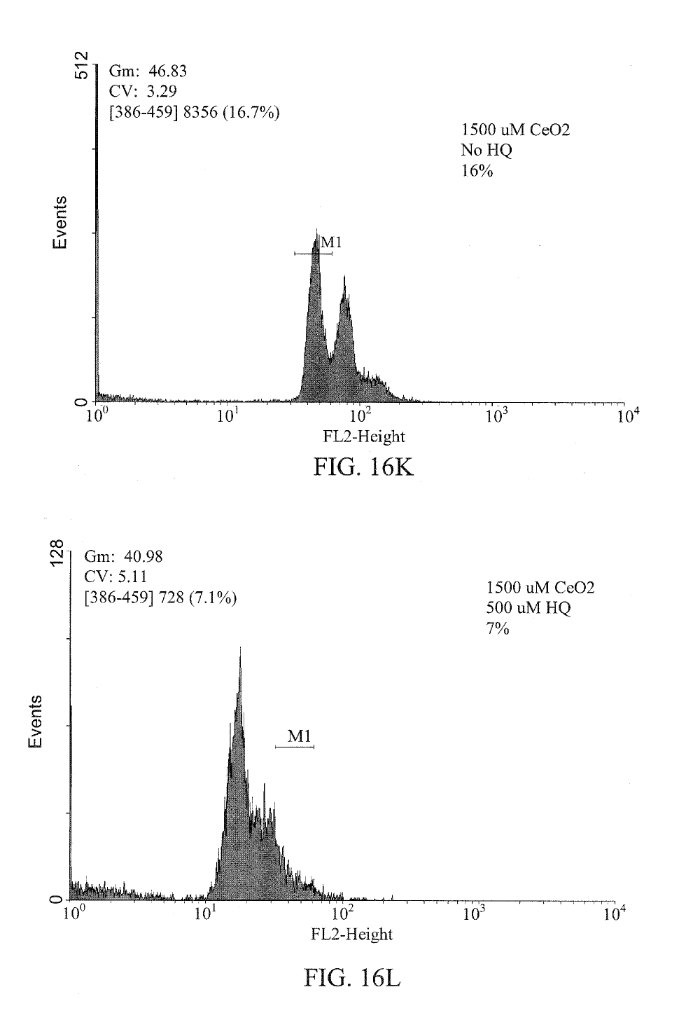

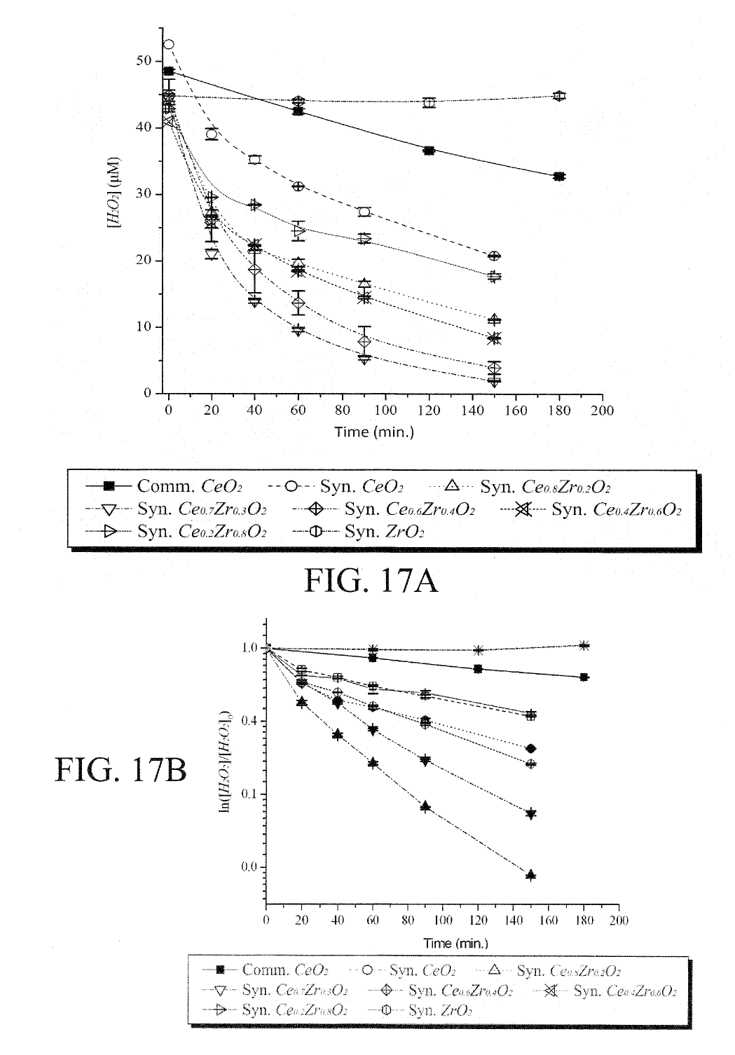

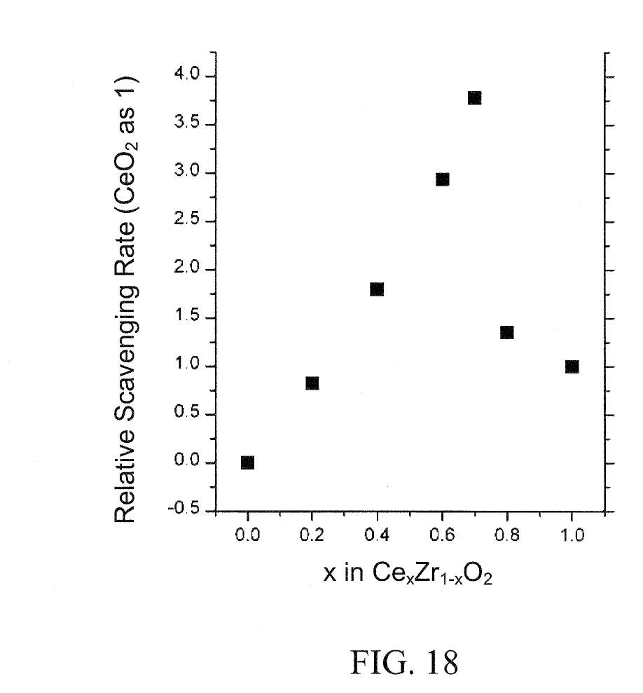

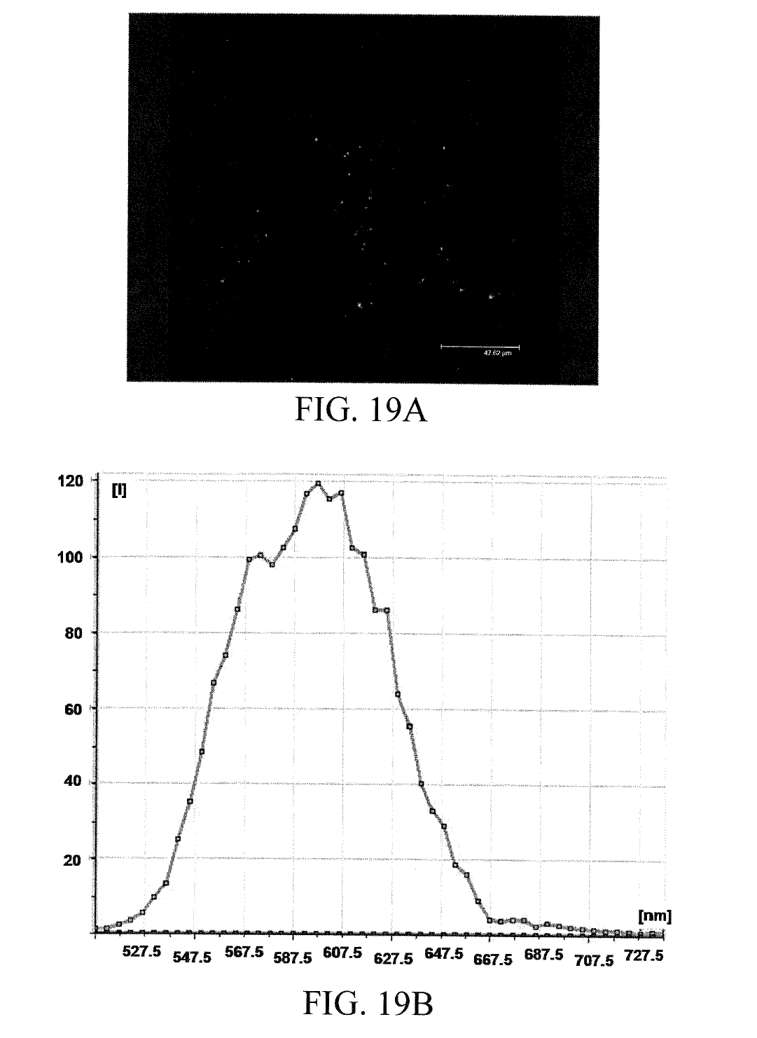

To assess the protective effect against H₂O₂ exposure, βTC-tet cells were loaded with lecithin-coated CeO₂ nanoparticles for 4 days (similarly to the way cells were prepared for the serum, deprivation experiment described above) and then exposed for 24 hours to either 50 or 100 μM H₂O₂. Unlike the serum deprivation experiment, where βTC-tet cells were exposed to media containing various concentrations of CeO₂ nanoparticles, in the H₂O₂ experiment, cells were exposed to media containing only 100 nM CeO₂. FIG. 9 shows the percent viable cells measured following a 24 hour exposure to H₂O₂. The data show that exposure to 100 μM H₂O₂ reduces the cell viability to less than 10% without an obvious beneficial effect by the nanoparticles. However, exposure to 50 μM H₂O₂ was not as detrimental to the cells resulted in cell viability of 70% for cells that where not exposed to CeO₂ nanoparticles and 80% for cell exposed to CeO₂ nanoparticles. This small “protection” was not statistically significant (p<0.09), but illustrates a potential that will be explored in the proposed experiments. One potential cause for this lack of statistical significance is the low CeO₂ concentration (100 nM) in the media and the subsequent intracellular cerium concentration detected by ICP.

To explore the importance of CeO₂ concentration, βTC-tet and human islets were exposed for up to 3 days to media containing 50, 100 or 200 μM citrate-coated CeO₂ nanoparticles. At the end of this incubation the cells/islets were exposed for 2 hours to media containing 50 μM H₂O₂. The efficacy of CeO₂ nanoparticles to scavenge ROS was assessed by measuring the concentration of intracellular ROS. This was achieved by measuring the fluorescent signal intensity of DCF (2,7-dichlorodihydrofluoresceindiacetate) as previously described [26]. FIG. 10 is a bar graph depicting the intracellular concentration of ROS in human islets for the various combinations of examined. The fluorescent signal detected from DCF was divided by the fluorescent signal derived from propidium iodine (PI). This was done to normalize the DCF signal to the number of cells under observation. Hence the data are presented as the ratio of DCF/PI. The data show that exposing human islets to media containing CeO₂ nanoparticles at 50 μM did not appreciably reduce the intracellular concentration of ROS. However, increasing the extracellular CeO₂ concentration from 50 to 100 or 200 μM caused an appreciable reduction in intracellular ROS concentration. More remarkable is the effect that the CeO₂ nanoparticles had when the islets were exposed to H₂O₂. Specifically, in the absence of CeO₂ nanoparticles islets exposed to H₂O₂ showed an elevation in the intracellular ROS concentration (control+H₂O₂). Conversely, islets pre-incubated with media containing 50 or 100 μM CeO₂ nanoparticles caused a reduction in intracellular ROS concentration but the levels were still elevated compared to islets that were not treated with H₂O₂. Finally, when islets were incubated with media containing 200 μM of ceria nanoparticles, the intracellular concentration of ROS was the same whether the islets were treated with H₂O₂ or not (200 μm Ce vs 200 μm Ce+H₂O₂). These patterns were also observed, in βTC-tet cells. This latest experiment is highly significant because: (a) it demonstrates that, to achieve a reduction in intracellular ROS concentration, larger quantities of ceria nanoparticles are needed, and (b) in addition to the model insulinoma cell lines, mammalian islets are also responsive to the beneficial effect of CeO₂ nanoparticles. The ICP and TEM analysis of the samples that were collected from this experiment is ongoing.

Overall, these data show that CeO₂ nanoparticles incorporate into insulin secreting cells and do not affect either their growth or metabolic activity. Furthermore, there is credible evidence supporting the hypothesis that CeO₂ based nanoparticles can be used to scavenge reactive oxygen species (ROS) in mammalian islets.

### Example 3

### Scavenging Role of Ceria Nanoparticles in Murine Islets

Further studies focus on the findings that certain nanoparticles (cerium oxide) dramatically increased the longevity of neuronal cells in vitro.

Materials and Methods

Ceria Nanoparticles. The ceria nanoparticles have and will continue to be engineered by a microemulsion method that ensures monodisperse nanoparticles with control in diameter ranging from 1-100 nm. The inventors will synthesize two sets of particle sizes 1-5 nm and 10-20 nm [10]. Each set will be treated at three different temperatures to control the stoichiometry (Ce³⁺/Ce⁴⁺ ratio) of the ceria nanoparticles [11]. The particles will be characterized with high resolution (0.014 nm) scanning transmission electron microscopy with elemental contrast (STEM-z) for size and electronic properties (selected area diffraction (SAD) and light scattering techniques for their ensemble properties, as well as Fourier Transform Infrared Spectroscopy (FTIR) and zeta-potential for the surface properties. Furthermore, the heat treatments impact on variation in stoichiometry of Ce³⁺/Ce⁴⁺ and the Ce³⁺/Ce⁴⁺ ratio will be measured by XPS [11,12]. The bandgap of the synthesized material will be characterized using differential reflectometry and photoluminescence spectroscopy [13]. Other techniques for quantitative characterization are available.

Mouse islet isolation and transplantation. Murine islets will be obtained. Islets will be hand picked, with dithizone staining utilized to determine purity [14]. Additional procedures associated with islet viability and function, representing standard operations of procedure, will be performed.

Culturing of purified mouse islets in the presence ceria nanoparticles. The initial series of experiments will involve the addition of ceria nanoparticles after islet isolation, as a culture supplement. An array of dosages ranging from 0.3 nM-30 nM dose (and a non-treated control culture) will be administered to the cultures.

Determination of islet viability. DNA content has been used as an indirect measure of cell mass, since the clustered nature of the islets, together with the non-endocrine contaminants, makes direct counting inappropriate [15]. DNA content will be measured in samples with and without ceria nanoparticle treatment. Islet viability will be determined by simultaneous staining of live and dead cells using a two-color fluorescence assay [16]. The percentage of viable and non-viable islets will also be estimated in both the treated and control cultures.

Assessment of reactive oxygen species (ROS) scavenging potential. To assess the ROS scavenging ability of ceria and other nanoparticles on islets, four experiments will be performed per type of nanoparticle synthesized. In the first experiments, the nanoparticles will be tested by themselves in the presence of 100 μM H₂O₂. In the second set of experiments, murine islets will be cultured in the presence of 100 μM H₂O₂ to enhance the presence of ROS. Subsequent to an overnight exposure, the islets will be separated into control and cerial treated groups and the presence of H₂O₂ islet viability will be assessed temporarily over a period of 7 days. H₂O₂ will be determined with commercially available fluorescent kits from Molecular Probes. In the third set of experiments, islets will be cultured in the presence and absence of ceria for 7 days. As the end of this incubation, all islets will be exposed to 100 μM H₂O₂ overnight. At the end of this treatment period, islet viability, apoptosis and H₂O₂ levels will be assessed. Finally, in the fourth set of experiments, islets will be cultured in the presence and absence of ceria and without H₂O₂ treatment to determine native benefit of ceria.

Assessment of islet function. The effects of the synthesized nanoparticles on islet viability and function will be assessed by using the second, third and fourth experiments described in the previous paragraph. The specific protocol that will be uses to assess metabolic and secretory function of islets is based on the protocol by Oca-Cossio et al. [9] that is described above with βTC-tet cells.

Immunohistochemistry. Samples will be fixed using standard procedures and stained for immunoreactive insulin, glucagon, somatostatin, CK19, and amylase and the percentage of positive cells will be counted in both control and ceria nanoparticle treated groups.

Alternative Methods. If nanoparticles do not show the expected enhancement, surface modifications to the particles will be carried out, using techniques that have been carried out with core-shell particles and smart nanoparticles, for example. Furthermore, a variety of semiconductor nanoparticles with similar electronic features can be synthesized (i.e., doped TiO₂, modified-fullerenes, doped ZnO and more) [3,17-20]. Such nanoparticles will be synthesized, characterized and tested for viability as given above. Synthetic procedures will build on published methods based on reversed micellar dispersions using protein or other surfactants, sol-gel chemistry or gas-phase synthesis.

TABLE 1
Examples of Target Cells

```
Keratinizing Epithelial Cells
keratinocyte of epidermis
basal cell of epidermis
keratinocyte of fingernails and toenails
basal cell of nail bed
hair shaft cells
    medullary
    cortical
    cuticular
hair-root sheath cells
    cuticular
    of Huxley's layer
    of Henle's layer
    external
hair matrix cell
Cells of Wet Stratified Barrier Epithelia
surface epithelial cell of stratified squamous epithelium of cornea
tongue, oral cavity,
esophagus, anal canal, distal urethra, vagina
basal cell of these epithelia
cell of urinary epithelium
Epithelial Cells Specialized for Exocrine Secretion
cells of salivary gland
    mucous cell
    serous cell
cell of von Ebner's gland in tongue
cell of mammary gland, secreting milk
cell of lacrimal gland, secreting tears
cell of ceruminous gland of ear, secreting wax
cell of eccrine sweat gland, secreting glycoproteins
cell of eccrine sweat gland, secreting small molecules
cell of apocrine sweat gland
cell of gland of Moll in eyelid
cell of sebaceous gland, secreting lipid-rich sebum
cell of Bowman's gland in nose
cell of Brunner's gland in duodenum, secreting alkaline solution of
mucus and enzymes
cell of seminal vesicle, secreting components of seminal fluid,
including fructose
cell of prostate gland, secreting other components of seminal fluid
cell of bulbourethral gland, secreting mucus
cell of Bartholin's gland, secreting vaginal lubricant
cell of gland of Littre, secreting mucus
cell of endometrium of uterus, secreting mainly carbohydrates
isolated goblet cell of respiratory and digestive tracts, secreting mucus
mucous cell of lining of stomach
zymogenic cell of gastric gland, secreting pepsinogen
oxyntic cell of gastric gland, secreting HCl
acinar cell of pancreas, secreting digestive enzymes and bicarbonate
Paneth cell of small intestine, secreting lysozyme
type II pneumocyte of lung, secreting surfactant
Clara cell of lung
Cells Specialized for Secretion of Hormones
cells of anterior pituitary, secreting
    growth hormone
    follicle-stimulating hormone
    luteinizing hormone
    prolactin
    adrenocorticotropic hormone
    thyroid-stimulating hormone
cell of intermediate pituitary, secreting melanocyte-stimulating hormone
cells of posterior pituitary, secreting
    oxytocin
    vasopressin
cells of gut and respiratory tract, secreting
    serotonin
    endorphin
    somatostatin
    gastrin
    secretin
    cholecystokinin
    insulin
    glucagons
    bombesin
cells of thyroid gland, secreting
    thyroid hormone
    calcitonin
cells of parathyroid gland, secreting
    parathyroid hormone
    oxyphil cell
cells of adrenal gland, secreting
    epinephrine
    norepinephrine
    steroid hormones
        mineralocorticoids
        glucocorticoids
cells of gonads, secreting
    testosterone
    estrogen
    progesterone
cells of juxtaglomerular apparatus of kidney
    juxtaglomerular cell
    macula densa cell
    peripolar cell
    mesangial cell
Epithelial Absorptive Cells in Gut, Exocrine Glands, and Urogenital Tract
brush border cell of intestine
striated duct cell of exocrine glands
gall bladder epithelial cell
brush border cell of proximal tubule of kidney
distal tubule cell of kidney
nonciliated cell of ductulus efferens
epididymal principal cell
epididymal basal cell
Cells Specialized for Metabolism and Storage
hepatocyte
fat cells (e.g., adipocyte)
    white fat
    brown fat
    lipocyte of liver
Epithelial Cells Serving Primarily a Barrier Function, Lining the
Lung, Gut,
Exocrine Glands, and Urogenital Tract
type I pneumocyte
pancreatic duct cell
nonstriated duct cell of sweat gland, salivary gland, mammary gland, etc.
parietal cell of kidney glomerulus
podocyte of kidney glomerulus
cell of thin segment of loop of Henle
collecting duct cell
duct cell of seminal vesicle, prostate gland, etc.
Epithelial Cells Lining Closed Internal Body Cavities
vascular endothelial cells of blood vessels and lymphatics
(e.g., microvascular cell)
    fenestrated
    continuous
    splenic
synovial cell
serosal cell
squamous cell lining perilymphatic space of ear
cells lining endolymphatic space of ear
    squamous cell
    columnar cells of endolymphatic sac
        with microvilli
        without microvilli
    “dark” cell
    vestibular membrane cell
    stria vascularis basal cell
    stria vascularis marginal cell
    cell of Claudius
    cell of Boettcher
choroid plexus cell
squamous cell of pia-arachnoid
cells of ciliary epithelium of eye
    pigmented
    nonpigmented
corneal “endothelial” cell
Ciliated Cells with Propulsive Function
of respiratory tract
of oviduct and of endometrium of uterus
of rete testis and ductulus efferens
of central nervous system
Cells Specialized for Secretion of Extracellular Matrix
epithelial:
    ameloblast
    planum semilunatum cell of vestibular apparatus of ear
    interdental cell of organ of Corti
nonepithelial:
    fibroblasts
    pericyte of blood capillary (Rouget cell)
    nucleus pulposus cell of intervertebral disc
    cementoblast/cementocyte
    odontoblast/odontocyte
    chondrocytes
        of hyaline cartilage
        of fibrocartilage
        of elastic cartilage
    osteoblast/osteocyte
    osteoprogenitor cell
    hyalocyte of vitreous body of eye
    stellate cell of perilymphatic space of ear
Contractile Cells
skeletal muscle cells
    red
    white
    intermediate
    muscle spindle-nuclear bag
    muscle spindle-nuclear chain
    satellite cell
heart muscle cells
    ordinary
    nodal
    Purkinje fiber
    Cardiac valve tissue
smooth muscle cells
myoepithelial cells:
    of iris
    of exocrine glands
Cells of Blood and Immune System
red blood cell (erythrocyte)
megakaryocyte
macrophages
    monocyte
    connective tissue macrophage
    Langerhan's cell
    osteoclast
    dendritic cell
    microglial cell
neutrophil
eosinophil
basophil
mast cell
plasma cell
T lymphocyte
    helper T cell
    suppressor T cell
    killer T cell
B lymphocyte
    IgM
    IgG
    IgA
    IgE
killer cell
stem cells and committed progenitors for the blood and immune system
Sensory Transducers
photoreceptors
    rod
    cones
        blue sensitive
        green sensitive
        red sensitive
hearing
    inner hair cell of organ of Corti
    outer hair cell of organ of Corti
acceleration and gravity
    type I hair cell of vestibular apparatus of ear
    type II hair cell of vestibular apparatus of ear
taste
    type II taste bud cell
smell
    olfactory neuron
    basal cell of olfactory epithelium
blood pH
    carotid body cell
        type I
        type II
touch
    Merkel cell of epidermis
    primary sensory neurons specialized for touch
temperature
    primary sensory neurons specialized for temperature
        cold sensitive
        heat sensitive
pain
    primary sensory neurons specialized for pain
configurations and forces in musculoskeletal system
    proprioceptive primary sensory neurons
Autonomic Neurons
cholinergic
adrenergic
peptidergic
Supporting Cells of Sense Organs and of Peripheral Neurons
supporting cells of organ of Corti
    inner pillar cell
    outer pillar cell
    inner phalangeal cell
    outer phalangeal cell
    border cell
    Hensen cell
supporting cell of vestibular apparatus
supporting cell of taste bud
supporting cell of olfactory epithelium
Schwann cell
satellite cell
enteric glial cell
Neurons and Glial Cells of Central Nervous System
neurons
glial cells
    astrocyte
    oligodendrocyte
Lens Cells
anterior lens epithelial cell
lens fiber
Pigment Cells
melanocyte
retinal pigmented epithelial cell
iris pigment epithelial cell
Germ Cells
oogonium/oocyte
spermatocyte
Spermatogonium
blast cells
fertilized ovum
Nurse Cells
ovarian follicle cell
Sertoli cell
thymus epithelial cell (e.g., reticular cell)
placental cell
```

All patents, patent applications, provisional applications, and publications referred to or cited herein, supra or infra, are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application.

### REFERENCES

[1] Rzigalinski, B. A. et al. Faseb J., 2003, 17(4):A606-A606, Part 1, Suppl. S.
[2] Chen, J. et al. Invest. Ophthalmology & Vis. Sci., 2005, 46:186 Suppl. S.
[3] Moghimi, S. M. et al. Faseb J., 2005, 19(3):311-330.
[4] Couzin, J. Science, 2004, 306:34-36.
[5] Chiang, Y. M. et al. J. Electroceramics, 1997, 1:7-14.
[6] Pileggi, A. et al. Diabetes, 2001, 50:1983-1991.
[7] Bottino, R. et al. Diabetes, 2002, 51:2562-2567.
[8] Huang, B. et al. Free Radical Bio. and Med., 2003, 35(7):805-813.
[9] Oca-Cossio, J. et al. Biochem. Biophys. Res. Comm., 2004, 319:569-575.
[10] Patil, S. et al. J. Nanoparticle Res., 2002, 4(5):433-438.
[11] Patina, Y. et al. Key Engineering Mat., 2003, 253:225-242.
[12] Zhang, F. et al. Surface Science, 2004, 563(1-3):74-82.
[13] Hummel, R. E. Surface and Interface Analysis, 1988, 12:11-14.
[14] Latif, Z. A. et al. Transplantation, 1998, 25:827-830.
[15] Ling, Z. and Pipeleers, D. G. J. Clin. Invest., 1996, 98:2805-2812.
[16] Lorenzo, A. et al. Nature, 1994, 26:756-760.
[17] Sigmund, W. and Kim, H. “Modification of Carbon Nanotubes” in Encyclopedia for Nanotechnology and Nanoscience, Ed. by H. Nalwa, 2004, 5:619-631.
[18] Sung-Hwan, L. et al. Coll. Surf. B, Bioint., 2005, 40(2):93-98.
[19] Bumsu, K. and Sigmund, W. Langmuir, 2004, 20(19):8239-8242.
[20] Sigmund, W. and Milz, C. J. Nanosci Nanotech., 2004, 4(3).

We claim:

1. A method for enhancing the survival and/or viability of cells, comprising contacting the cells with metal oxide nanoparticles having free radical scavenging activity.

2. The method of claim 1, wherein said contacting is carried out in vitro.

3. The method of claim 1, wherein said contacting is carried out in vivo.

4. The method of claim 1, wherein said contacting is carried out in vitro, and the cells are subsequently administered to a mammalian subject.

5. The method of claim 4, wherein the cells were removed from the subject prior to said contacting.

5. The method of claim 1, wherein the nanoparticles are cerium oxide nanoparticles.

6. The method of claim 1, wherein the nanoparticles are citrate-coated cerium oxide nanoparticles.

7. The method of claim 1, wherein the cells are pancreatic cells or skin cells.

8. The method of claim 1, wherein the metal oxide is selected from the group consisting of zinc oxide yttrium oxide, zirconium oxide, bismuth oxide, and cadmium oxide.

9. The method of claim 1, wherein the nanoparticles are doped, fluorescent nanoparticles.

10. The method of claim 1, wherein the nanoparticles are Erbium-doped nanoparticles.

11. The method of claim 1, wherein the nanoparticles are Erbium-doped cerium oxide nanoparticles.

12. The method of claim 1, wherein the nanoparticles are less than 20 nanometers in diameter.

13. A citrate-coated metal oxide nanoparticle.

14. A doped, fluorescent, metal oxide nanoparticle.

15. The nanoparticle of claim 14, wherein the nanoparticle is Erbium-doped.

16. A composition comprising cells and metal oxide nanoparticles.

17. A cell preservation fluid, comprising metal oxide nanoparticles and one or more cell preservation agents.
